(19) United States
(12) Patent Application Publication
(10) Pub. No.: US 2024/0252487 A1
(43) Pub. Date: Aug. 1, 2024

(54) METHODS OF TREATING MULTIPLE MYELOMA USING COMBINATION THERAPY

(71) Applicants: Celgene Corporation, Summit, NJ (US); Bristol-Myers Squibb Company, Princeton, NJ (US)

(72) Inventors: Paulo Cesar MACIAG, Summit, NJ (US); Teresa PELUSO, Rome (IT)

(21) Appl. No.: 18/563,037

(22) PCT Filed: May 26, 2022
(86) PCT No.: PCT/US2022/031055

§ 371 Date: Nov. 21, 2023

Related U.S. Application Data
(60) Provisional application No. 63/194,026, filed on May 27, 2021.

(51) Int. Cl. A61K 31/496; A61K 31/573; A61K 39/395; A61P 35/00

(52) U.S. Cl. CPC A61K 31/496; A61K 31/573; A61K 39/3955; A61P 35/00

(57) ABSTRACT

Provided herein are methods of using (S)-4-(4-(4-(((2-(2,6-dioxopiperidin-3-yl)-1-oxoisoin-dolin-4-yl)oxy)methyl)benzyl)piperazin1-yl)-3-fluorobenzonitrile, or an enantiomer, a mixture of enantiomers, a tautomer, or a pharmaceutically acceptable salt thereof, in combination with a second active agent provided herein for treating, preventing or managing multiple myeloma.

### CROSS-REFERENCE TO RELATED APPLICATIONS

This application claims priority to U.S. Provisional Application No. 63/194,026, filed on May 27, 2021, the entirety of which is incorporated herein by reference.

### FIELD

Provided herein are methods of using (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, or a pharmaceutically acceptable salt thereof, in combination with a second active agent provided herein for treating, preventing or managing multiple myeloma.

### BACKGROUND

Multiple myeloma (MM) is a cancer of plasma cells in the bone marrow. Normally, plasma cells produce antibodies and play a key role in immune function. However, uncontrolled growth of these cells leads to bone pain and fractures, anemia, infections, and other complications. Multiple myeloma is the second most common hematological malignancy, although the exact causes of multiple myeloma remain unknown. Multiple myeloma causes high levels of proteins in the blood, urine, and organs, including but not limited to M-protein and other immunoglobulins (antibodies), albumin, and beta-2-microglobulin, except in some patients (estimated at 1% to 5%) whose myeloma cells do not secrete these proteins (termed non-secretory myeloma). M-protein, short for monoclonal protein, also known as paraprotein, is a particularly abnormal protein produced by the myeloma plasma cells and can be found in the blood or urine of almost all patients with multiple myeloma, except for patients who have non-secretory myeloma or whose myeloma cells produce immunoglobulin light chains with heavy chain.

Skeletal symptoms, including bone pain, are among the most clinically significant symptoms of multiple myeloma. Malignant plasma cells release osteoclast stimulating factors (including IL-1, IL-6 and TNF) which cause calcium to be leached from bones causing lytic lesions; hypercalcemia is another symptom. The osteoclast stimulating factors, also referred to as cytokines, may prevent apoptosis, or death of myeloma cells. Fifty percent of patients have radiologically detectable myeloma-related skeletal lesions at diagnosis. Other common clinical symptoms for multiple myeloma include polyneuropathy, anemia, hyperviscosity, infections, and renal insufficiency.

Current multiple myeloma therapy may involve one or more of surgery, stem cell transplantation, chemotherapy, immune therapy, and/or radiation treatment to eradicate multiple myeloma cells in a patient. All of the current therapy approaches pose significant drawbacks for the patient.

In the last decade, novel therapeutic agents, in particular immunomodulatory drugs such as lenalidomide and pomalidomide, significantly increased the response rates and prolonged progression free survival (PFS) and overall survival (OS) in multiple myeloma patients. However, persistent levels of residual disease that are below the sensitivity of bone marrow (BM) morphology, protein electrophoresis with immunofixation, and light chain quantitation exists in many patients with multiple myeloma, even after these patients have achieved complete response (CR), and will eventually cause relapse of the disease. Minimal residual disease (MRD) in myeloma is an independent predictor of progression-free survival (PFS) and is under consideration as a surrogate trial endpoint to improve the identification of effective treatments, particularly for frontline trials, which now require 5 to 10 years of follow-up to identify survival differences. Monitoring minimal residual disease (MRD) in patients with multiple myeloma thus provides prognostic value in predicting PFS and OS and making treatment decisions. The detection of minimal residual disease (MRD) in myeloma can use a 0.01% threshold (10⁻⁴) after treatment, i.e., having 10⁻⁴ cells or fewer multiple myeloma cells as a proportion of total bone marrow mononuclear cells is considered MRD-negative, and having 10⁻⁴ cells or higher MRD-positive. The 10⁻⁴ MRD threshold was originally based on technical capability, but quantitative MRD detection is now possible at 10⁻⁵ by flow cytometry and 10⁻⁶ by high-throughput sequencing. (Rawstron et al., Blood 2015; 125(12): 1932-1935). Methods for measuring MRD include DNA sequencing of VDJ, polymerase chain reaction (PCR) (including allele specific PCR, ASO PCR) and multiparameter flow cytometry (MPF). Assays for MRD, e.g., based on clonotype profile measurement are also described in U.S. Pat. No. 8,628,927, to Faham et al., which is incorporated herein by reference.

There exists a significant need for safe and effective compounds and methods for treating, preventing and managing multiple myeloma, including for patients whose multiple myeloma is newly diagnosed or refractory to standard treatments, while reducing or avoiding the toxicities and/or side effects associated with the conventional therapies.

Citation or identification of any reference in Section 2 of this application is not to be construed as an admission that the reference is prior art to the present application.

### SUMMARY

Provided herein are methods of using (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, or a pharmaceutically acceptable salt thereof, in combination with a second active agent provided herein for treating, preventing or managing multiple myeloma. In one embodiment, the second agent is (i) a combination of bortezomib and dexamethasone; (ii) a combination of daratumumab and dexamethasone; (iii) a combination of carfilzomib and dexamethasone; (iv) a combination of elotuzumab and dexamethasone; or (v) a combination of isatuximab and dexamethasone.

In one embodiment, provided herein is a method of treating multiple myeloma, comprising administering to a subject in need thereof a therapeutically effective amount of a compound of Formula (I):

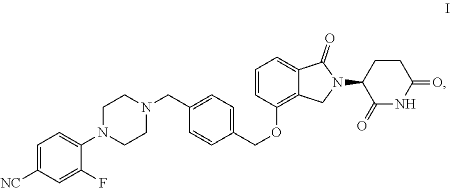 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with elotuzumab and dexamethasone.

In one embodiment, provided herein is a method of treating multiple myeloma, comprising administering to a subject in need thereof a therapeutically effective amount of a compound of Formula (I):

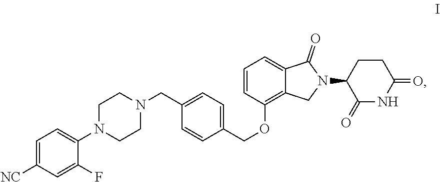 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with isatuximab and dexamethasone.

In one embodiment, the multiple myeloma is relapsed or refractory multiple myeloma (RRMM). In one embodiment, the multiple myeloma is newly diagnosed multiple myeloma (NDMM).

In one embodiment, provided herein is the combination of compounds provided herein for use in methods of treating the diseases provided herein.

These and other aspects of the subject matter described herein will become evident upon reference to the following detailed description.

### DETAILED DESCRIPTION

### Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein, and in the specification and the accompanying claims, the indefinite articles “a” and “an” and the definite article “the” include plural as well as single referents, unless the context clearly indicates otherwise.

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

As used herein, the phrase “and/or” as used in a phrase such as “A and/or B” herein is intended to include both A and B; A or B; A (alone); and B (alone). Likewise, the phrase “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethyl-benzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, fumarates and organic sulfonates.

Unless specifically stated otherwise, where a compound may assume alternative tautomeric, regioisomeric and/or stereoisomeric forms, all alternative isomers are intended to be encompassed within the scope of the claimed subject matter. For example, where a compound can have one of two tautomeric forms, it is intended that both tautomers be encompassed herein.

Thus, the compounds provided herein may be enantiomerically pure, or be stereoisomeric or diastereomeric mixtures. As used herein and unless otherwise indicated, the term “stereoisomerically pure” means a composition that comprises one stereoisomer of a compound and is substantially free of other stereoisomers of that compound. For example, a stereoisomerically pure composition of a compound having one chiral center will be substantially free of the opposite enantiomer of the compound. A stereoisomerically pure composition of a compound having two chiral centers will be substantially free of other diastereomers of the compound. A typical stereoisomerically pure compound comprises greater than about 80% by weight of one stereoisomer of the compound and less than about 20% by weight of other stereoisomers of the compound, more preferably greater than about 90% by weight of one stereoisomer of the compound and less than about 10% by weight of the other stereoisomers of the compound, even more preferably greater than about 95% by weight of one stereoisomer of the compound and less than about 5% by weight of the other stereoisomers of the compound, and most preferably greater than about 97% by weight of one stereoisomer of the compound and less than about 3% by weight of the other stereoisomers of the compound. A stereoisomerically pure compound as used herein comprises greater than about 80% by weight of one stereoisomer of the compound, more preferably greater than about 90% by weight of one stereoisomer of the compound, even more preferably greater than about 95% by weight of one stereoisomer of the compound, and most preferably greater than about 97% by weight of one stereoisomer of the compound. As used herein and unless otherwise indicated, the term “stereoisomerically enriched” means a composition that comprises greater than about 60% by weight of one stereoisomer of a compound, preferably greater than about 70% by weight, more preferably greater than about 80% by weight of one stereoisomer of a compound. As used herein and unless otherwise indicated, the term “enantiomerically pure” means a stereoisomerically pure composition of a compound having one chiral center. Similarly, the term “stereoisomerically enriched” means a stereoisomerically enriched composition of a compound having one chiral center. As used herein, stereoisomeric or diastereomeric mixtures means a composition that comprises more than one stereoisomer of a compound. A typical stereoisomeric mixture of a compound comprises about 50% by weight of one stereoisomer of the compound and about 50% by weight of other stereoisomers of the compound, or comprises greater than about 50% by weight of one stereoisomer of the compound and less than about 50% by weight of other stereoisomers of the compound, or comprises greater than about 45% by weight of one stereoisomer of the compound and less than about 55% by weight of the other stereoisomers of the compound, or comprises greater than about 40% by weight of one stereoisomer of the compound and less than about 60% by weight of the other stereoisomers of the compound, or comprises greater than about 35% by weight of one stereoisomer of the compound and less than about 65% by weight of the other stereoisomers of the compound.

It is to be understood that the compounds provided herein may contain chiral centers. Such chiral centers may be of either the (R) or (S) configuration, or may be a mixture thereof. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

Optically active (+) and (−), (R)- and (S)-, or (D)- and (L)-isomers may be prepared using chiral synthons or chiral reagents, or resolved using conventional techniques, such as chromatography on a chiral stationary phase.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

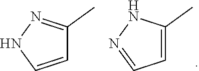

As used herein, an “isotopolog” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., multiple myeloma therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues of Compound 1, Compound 2 or Compound 3 are deuterium, carbon-13, or nitrogen-15 enriched compounds. In some embodiments, isotopologues provided herein are deuterium enriched compounds. In some embodiments, isotopologues provided herein are deuterium enriched compounds, where the deuteration occurs on the chiral center.

It should be noted that if there is a discrepancy between a depicted structure and a name for that structure, the depicted structure is to be accorded more weight.

As used herein “multiple myeloma” refers to hematological conditions characterized by malignant plasma cells and includes the following disorders: monoclonal gammopathy of undetermined significance (MGUS); low risk, intermediate risk, and high risk multiple myeloma; newly diagnosed multiple myeloma (including low risk, intermediate risk, and high risk newly diagnosed multiple myeloma); transplant eligible and transplant ineligible multiple myeloma; smoldering (indolent) multiple myeloma (including low risk, intermediate risk, and high risk smouldering multiple myeloma); active multiple myeloma; solitary plasmacytoma; extramedullary plasmacytoma; plasma cell leukemia; central nervous system multiple myeloma; light chain myeloma; non-secretory myeloma; Immunoglobulin D myeloma; and Immunoglobulin E myeloma; and multiple myeloma characterized by genetic abnormalities, such as Cyclin D translocations (for example, t(11;14)(q13;q32); t(6; 14)(p21;32); t(12;14)(p13;q32); or t(6;20);); MMSET translocations (for example, t(4;14)(p16;q32)); MAF translocations (for example, t(14; 16)(q32;q32); t(20;22); t(16; 22)(q11;q13); or t(14;20)(q32;q11)); or other chromosome factors (for example, deletion of 17p13, or chromosome 13; del(17/17p), nonhyperdiploidy, and gain(1q)). In one embodiment, the multiple myeloma is characterized according to the multiple myeloma International Staging System (ISS). In one embodiment, the multiple myeloma is Stage I multiple myeloma as characterized by ISS (e.g., serum β2 microglobulin <3.5 mg/L and serum albumin ≥3.5 g/dL). In one embodiment, the multiple myeloma is Stage III multiple myeloma as characterized by ISS (e.g., serum β2 microglobulin >5.4 mg/L). In one embodiment, the multiple myeloma is Stage II multiple myeloma as characterized by ISS (e.g., not Stage I or III).

As used herein and unless otherwise indicated, the terms “treat,” “treating” and “treatment” refer to alleviating or reducing the severity of a symptom associated with the disease or condition being treated, for example, multiple myeloma.

The term “prevention” includes the inhibition of a symptom of the particular disease or disorder, for example multiple myeloma. In some embodiments, patients with familial history of multiple myeloma are candidates for preventive regimens. Generally, the term “preventing” refers to administration of the drug prior to the onset of symptoms, particularly to patients at risk of multiple myeloma.

As used herein and unless otherwise indicated, the term “managing” encompasses preventing the recurrence of the particular disease or disorder, such as multiple myeloma, in a patient who had suffered from it, lengthening the time a patient who had suffered from the disease or disorder remains in remission, reducing mortality rates of the patients, and/or maintaining a reduction in severity or avoidance of a symptom associated with the disease or condition being managed.

As used herein, “subject” or “patient” is an animal, typically a mammal, including a human, such as a human patient.

The term “relapsed” refers to a situation where patients who have had a remission of multiple myeloma after therapy have a return of myeloma cells and/or reduced normal cells in the marrow.

The term “refractory or resistant” refers to a circumstance where patients, even after intensive treatment, have residual myeloma cells and/or reduced normal cells in the marrow.

As used herein, “induction therapy” refers to the first treatment given for a disease, or the first treatment given with the intent of inducing complete remission in a disease, such as cancer. When used by itself, induction therapy is the one accepted as the best available treatment. If residual cancer is detected, patients are treated with another therapy, termed reinduction. If the patient is in complete remission after induction therapy, then additional consolidation and/or maintenance therapy is given to prolong remission or to potentially cure the patient.

As used herein, “consolidation therapy” refers to the treatment given for a disease after remission is first achieved. For example, consolidation therapy for cancer is the treatment given after the cancer has disappeared after initial therapy. Consolidation therapy may include radiation therapy, stem cell transplant, or treatment with cancer drug therapy. Consolidation therapy is also referred to as intensification therapy and post-remission therapy.

As used herein, “maintenance therapy” refers to the treatment given for a disease after remission or best response is achieved, in order to prevent or delay relapse. Maintenance therapy can include chemotherapy, hormone therapy or targeted therapy.

“Remission” as used herein, is a decrease in or disappearance of signs and symptoms of a cancer, for example, multiple myeloma. In partial remission, some, but not all, signs and symptoms of the cancer have disappeared. In complete remission, all signs and symptoms of the cancer have disappeared, although the cancer still may be in the body.

As used herein “transplant” refers to high-dose therapy with stem cell rescue. Hematopoietic (blood) or bone marrow stem cells are used not as treatment but to rescue the patient after the high-dose therapy, for example high dose chemotherapy and/or radiation. Transplant includes “autologous” stem cell transplant (ASCT), which refers to use of the patients' own stem cells being harvested and used as the replacement cells. In some embodiments, transplant also includes tandem transplant or multiple transplants.

As used herein, and unless otherwise specified, the terms “therapeutically effective amount” and “effective amount” of a compound refer to an amount sufficient to provide a therapeutic benefit in the treatment, prevention and/or management of a disease, for example multiple myeloma, or to delay or minimize one or more symptoms associated with the disease or disorder to be treated. The terms “therapeutically effective amount” and “effective amount” can encompass an amount that improves overall therapy, reduces or avoids symptoms or causes of disease or disorder, or enhances the therapeutic efficacy of another therapeutic agent.

The terms “co-administration” and “in combination with” include the administration of one or more therapeutic agents (for example, a compound provided herein and another anti-multiple myeloma agent, cancer agent or supportive care agent) either simultaneously, concurrently or sequentially with no specific time limits. In one embodiment, the agents are present in the cell or in the patient's body at the same time or exert their biological or therapeutic effect at the same time. In one embodiment, the therapeutic agents are in the same composition or unit dosage form. In another embodiment, the therapeutic agents are in separate compositions or unit dosage forms.

As used herein and unless otherwise specified, “a therapeutic agent” provided herein is not limited to a single therapeutic agent, and it can be, in certain embodiments, a combination of one or more different therapeutic agents. The one or more therapeutic agents can be administered in combination with each other as described herein. As used herein and unless otherwise specified, “a therapeutic agent” can be used interchangeably with “a therapeutic therapy”, and is not limited to a therapeutic substance. For example, a therapeutic agent can be a cancer treatment such as radiation therapy or CAR-T therapy.

An “cycling therapy” refers to a regimen or therapy that includes an administration period as described herein and optionally a rest period as described herein.

The term “administration period” as used herein refers to a period of time a subject is continuously or actively administered a compound or composition described herein.

The term “rest period” as used herein refers to a period of time, often following an administration period, where a subject is not administered a compound or composition described herein (e.g. discontinuation of treatment). In certain embodiments, a “rest period” refers to a period of time where a single agent is not administered to a subject or treatment using a particular compound is discontinued. In such embodiments, a second therapeutic agent (e.g., a different agent than the compound or composition administered in the previous administration period) can be administered to the subject.

The term “supportive care agent” refers to any substance that treats, prevents or manages an adverse effect from treatment with Compound 1, Compound 2 or Compound 3, or an enantiomer or a mixture of enantiomers, tautomers, isotopolog or a pharmaceutically acceptable salt thereof.

The term “biological therapy” refers to administration of biological therapeutics such as cord blood, stem cells, growth factors and the like.

In the context of a cancer, such as multiple myeloma, inhibition may be assessed by inhibition of disease progression, inhibition of tumor growth, reduction of primary tumor, relief of tumor-related symptoms, inhibition of tumor secreted factors, delayed appearance of primary or secondary tumors, slowed development of primary or secondary tumors, decreased occurrence of primary or secondary tumors, slowed or decreased severity of secondary effects of disease, arrested tumor growth and regression of tumors, increased Time To Progression (TTP), increased Progression Free Survival (PFS), increased Overall Survival (OS), among others. OS as used herein means the time from treatment onset until death from any cause. TTP, as used herein, means the time from treatment onset until tumor progression; TTP does not include deaths. In one embodiment, PFS means the time from treatment onset until tumor progression or death. In one embodiment, PFS means the time from the first dose of compound to the first occurrence of disease progression or death from any cause. In one embodiment, PFS rates will be computed using the Kaplan-Meier estimates. Event-free survival (EFS) means the time from treatment onset until any treatment failure, including disease progression, treatment discontinuation for any reason, or death. In one embodiment, overall response rate (ORR) means the percentage of patients who achieve a response. In one embodiment, ORR means the sum of the percentage of patients who achieve complete and partial responses. In one embodiment, ORR means the percentage of patients whose best response≥partial response (PR), according to the IMWG Uniform Response Criteria. In one embodiment, duration of response (DoR) is the time from achieving a response until relapse or disease progression. In one embodiment, DoR is the time from achieving a response≥partial response (PR) until relapse or disease progression. In one embodiment, DoR is the time from the first documentation of a response until to the first documentation of progressive disease or death. In one embodiment, DoR is the time from the first documentation of a response≥partial response (PR) until to the first documentation of progressive disease or death. In one embodiment, time to response (TTR) means the time from the first dose of compound to the first documentation of a response. In one embodiment, TTR means the time from the first dose of compound to the first documentation of a response≥partial response (PR). In the extreme, complete inhibition, is referred to herein as prevention or chemoprevention. In this context, the term “prevention” includes either preventing the onset of clinically evident cancer altogether or preventing the onset of a preclinically evident stage of a cancer. Also intended to be encompassed by this definition is the prevention of transformation into malignant cells or to arrest or reverse the progression of premalignant cells to malignant cells. This includes prophylactic treatment of those at risk of developing a cancer.

In certain embodiments, the treatment of multiple myeloma may be assessed by the International Uniform Response Criteria for Multiple Myeloma (IURC) (see Durie B G M, Harousseau J-L, Miguel J S, et al. International uniform response criteria for multiple myeloma. Leukemia, 2006; (10) 10: 1-7), using the response and endpoint definitions shown below:

| Response | |
|---|---|
| Subcategory | Response Criteria^{a} |
| sCR | CR as defined below plus |
| | Normal FLC ratio and |
| | Absence of clonal cells in bone marrow^{b} by |
| | immunohistochemistry or immunofluorescence^{c} |
| CR | Negative immunofixation on the serum and urine and |
| | Disappearance of any soft tissue plasmacytomas |
| | and <5% plasma cells in bone marrow^{b} |
| VGPR | Serum and urine M-protein detectable by immunofixation but |
| | not on electrophoresis or 90% or greater reduction in serum |
| | M-protein plus urine M-protein level <100 mg per 24 h |
| PR | ≥50% reduction of serum M-protein and reduction in 24-h |
| | urinary M-protein by ≥90% or to <200 mg per 24 h |
| | If the serum and urine M-protein are unmeasurable,^{d} a ≥50% |
| | decrease in the difference between involved and uninvolved |
| | FLC levels is required in place of the M-protein criteria |
| | If serum and urine M-protein are unmeasurable, and serum free |
| | light assay is also unmeasurable, ≥50% reduction in plasma |
| | cells is required in place of M-protein, provided baseline bone |
| | marrow plasma cell percentage was ≥30% |
| | In addition to the above listed criteria, if present at baseline, |
| | a ≥50% reduction in the size of soft tissue plasmacytomas is |
| | also required |
| SD (not | Not meeting criteria for CR, VGPR, PR or progressive disease |
| recommended for | |
| use as an indicator | |
| of response; stability | |
| of disease is best | |
| described by | |
| providing the time | |
| to progression | |
| estimates) | |

Abbreviations: CR, complete response; FLC, free light chain; PR, partial response; SD, stable disease; sCR, stringent complete response; VGPR, very good partial response.
^{a}All response categories require two consecutive assessments made at any time before the institution of any new therapy; all categories also require no known evidence of progressive or new bone lesions if radiographic studies were performed. Radiographic studies are not required to satisfy these response requirements.
^{b}Confirmation with repeat bone marrow biopsy not needed.
^{c}Presence/absence of clonal cells is based upon the κ/λ ratio. An abnormal κ/λ ratio by immunohistochemistry and/or immunofluorescence requires a minimum of 100 plasma cells for analysis. An abnormal ratio reflecting presence of an abnormal clone is κ/λ of >4:1 or <1:2.
^{d}Measurable disease defined by at least one of the following measurements: Bone marrow plasma cells ≥30%; Serum M-protein ≥1 g/dl (≥10 gm/l)[10 g/l]; Urine M-protein ≥200 mg/24 h; Serum FLC assay: Involved FLC level ≥10 mg/dl (≥100 mg/l); provided serum FLC ratio is abnormal.

As used herein, ECOG status refers to Eastern Cooperative Oncology Group (ECOG) Performance Status (Oken M, et al Toxicity and response criteria of the Eastern Cooperative Oncology Group. Am J Clin Oncol 1982; 5(6):649-655), as shown below:

| Score | Description |
|---|---|
| 0 | Fully active, able to carry on all pre-disease performance without |
| | restriction |
| 1 | Restricted in physically strenuous activity but ambulatory and able |
| | to carry out work of a light or sedentary nature, eg, light |
| | housework, office work. |
| 2 | Ambulatory and capable of all self-care but unable to carry out any |
| | work activities. Up and about more than 50% of waking hours. |
| 3 | Capable of only limited self-care, confined to bed or chair more |
| | than 50% of waking hours. |
| 4 | Completely disabled. Cannot carry on any self-care. Totally |
| | confined to bed or chair |
| 5 | Dead |

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In one embodiment, the terms “about” and “approximately,” when used in this context, contemplate a dose, amount, or weight percent within 30%, within 20%, within 15%, within 10%, or within 5%, of the specified dose, amount, or weight percent.

### Compounds

In one embodiment, the compound used in the methods provided herein is (S)-4-(4-(4-(((2-(2,6-dioxopiperidin-3-yl)-1-oxoisoindolin-4-yl)oxy)methyl)benzyl)piperazin-1-yl)-3-fluorobenzonitrile of the formula:

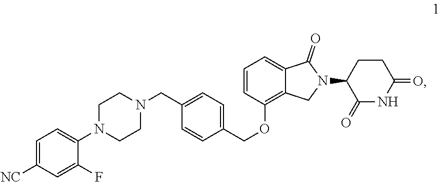 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof. (S)-4-(4-(4-(((2-(2,6-dioxopiperidin-3-yl)-1-oxoisoindolin-4-yl)oxy)methyl)benzyl)piperazin-1-yl)-3-fluorobenzonitrile is also referred herein as “Compound 1”.

In one embodiment, the compound used in the methods provided herein is (R)-4-(4-(4-(((2-(2,6-dioxopiperidin-3-yl)-1-oxoisoindolin-4-yl)oxy)methyl)benzyl)piperazin-1-yl)-3-fluorobenzonitrile (referred herein as “Compound 2”) of the formula:

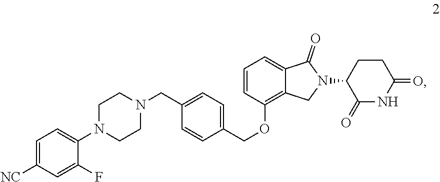 [SMILES: N#Cc1ccc(N2CCN(Cc3ccc(COc4cccc5c4CN([C@@H]4CCC(=O)NC4=O)C5=O)cc3)CC2)c(F)c1]

or a tautomer, isotopolog, or pharmaceutically acceptable salt thereof.

In one embodiment, the compound used in the methods provided herein is 4-(4-(4-(((2-(2,6-dioxopiperidin-3-yl)-1-oxoisoindolin-4-yl)oxy)methyl)benzyl)piperazin-1-yl)-3-fluorobenzonitrile (referred herein as “Compound 3”) of the formula:

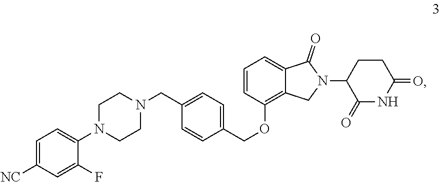 [SMILES: N#Cc1ccc(N2CCN(Cc3ccc(COc4cccc5c4CN(C4CCC(=O)NC4=O)C5=O)cc3)CC2)c(F)c1]

or a tautomer, isotopolog, or pharmaceutically acceptable salt thereof.

In one embodiment, Compound 1 (free base) is used in the methods provided herein. In one embodiment, a tautomer of Compound 1 is used in the methods provided herein. In one embodiment, an isotopolog of Compound 1 is used in the methods provided herein. In one embodiment, a pharmaceutically acceptable salt of Compound 1 is used in the methods provided herein. In one embodiment, a hydrobromide salt of Compound 1 is used in the methods provided herein. In one embodiment, a mono-hydrobromide salt of Compound 1 is used in the methods provided herein. Certain salts and polymorphic forms of Compound 1 are described in U.S. Patent Application Publication No. 2020-0216418, the entirety of which is incorporated herein by reference.

In one embodiment, Compound 2 is used in the methods provided herein. In one embodiment, a tautomer of Compound 2 is used in the methods provided herein. In one embodiment, an isotopolog of Compound 2 is used in the methods provided herein. In one embodiment, a pharmaceutically acceptable salt of Compound 2 is used in the methods provided herein.

In one embodiment, Compound 3 is used in the methods provided herein. In one embodiment, a tautomer of Compound 3 is used in the methods provided herein. In one embodiment, an isotopolog of Compound 3 is used in the methods provided herein. In one embodiment, a pharmaceutically acceptable salt of Compound 3 is used in the methods provided herein.

In one embodiment, isotopically enriched analogs of the compounds are used in the methods provided herein. In one embodiment, the isotopically enriched analogs of the compounds used in the methods provided herein include those described in U.S. Pat. No. 10,357,489, which is incorporated herein by reference in its entirety.

The synthesis and certain use of the compounds provided herein are described in U.S. Pat. No. 10,357,489 and U.S. Patent Application Publication No. 2020-0215060, the entirety of each of which is incorporated herein by reference.

### Methods of Treatment and Prevention

In one embodiment, provided herein are methods of using (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, or a pharmaceutically acceptable salt thereof, in combination with a second active agent provided herein for treating, preventing or managing multiple myeloma. In one embodiment, the second agent is (i) a combination of bortezomib and dexamethasone; (ii) a combination of daratumumab and dexamethasone; (iii) a combination of carfilzomib and dexamethasone; (iv) a combination of elotuzumab and dexamethasone; or (v) a combination of isatuximab and dexamethasone. In one embodiment, provided herein is (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, or a pharmaceutically acceptable salt thereof, for use in a method of treating, preventing or managing multiple myeloma, wherein the method comprises the additional administration of a second active agent provided herein. In one embodiment, provided herein is (S)-4-(4-(4-(((2-(2,6-dioxopiperidin-3-yl)-1-oxoisoindolin-4-yl)oxy)methyl)benzyl)piperazin-1-yl)-3-fluorobenzonitrile, or an enantiomer, a mixture of enantiomers, a tautomer, or a pharmaceutically acceptable salt thereof, for use in a method of treating multiple myeloma, wherein the method comprises the additional administration of a second active agent provided herein.

In one embodiment, provided herein is a method of treating multiple myeloma, comprising administering to a subject in need thereof a therapeutically effective amount of a compound of Formula (I):

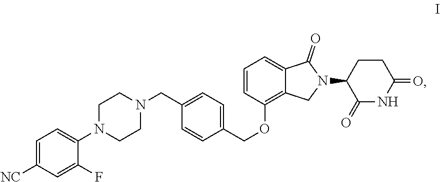 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with a second therapeutic agent, wherein the second therapeutic agent is: (i) a combination of bortezomib and dexamethasone; (ii) a combination of daratumumab and dexamethasone; (iii) a combination of carfilzomib and dexamethasone; (iv) a combination of elotuzumab and dexamethasone; or (v) a combination of isatuximab and dexamethasone. Unless otherwise specified, “a compound of Formula (I)” and “Compound 1” are used interchangeably herein.

In one embodiment, provided herein is a method of preventing multiple myeloma, comprising administering to a subject in need thereof a therapeutically effective amount of Compound 1, or an enantiomer, mixture of enantiomers, tautomer, isotopolog, or pharmaceutically acceptable salt thereof, in combination with a second therapeutic agent, wherein the second therapeutic agent is: (i) a combination of bortezomib and dexamethasone; (ii) a combination of daratumumab and dexamethasone; (iii) a combination of carfilzomib and dexamethasone; (iv) a combination of elotuzumab and dexamethasone; or (v) a combination of isatuximab and dexamethasone.

In one embodiment, provided herein is a method of managing multiple myeloma, comprising administering to a subject in need thereof a therapeutically effective amount of Compound 1, or an enantiomer, mixture of enantiomers, tautomer, isotopolog, or pharmaceutically acceptable salt thereof, in combination with a second therapeutic agent, wherein the second therapeutic agent is: (i) a combination of bortezomib and dexamethasone; (ii) a combination of daratumumab and dexamethasone; (iii) a combination of carfilzomib and dexamethasone; (iv) a combination of elotuzumab and dexamethasone; or (v) a combination of isatuximab and dexamethasone.

In one embodiment, the multiple myeloma is previously untreated multiple myeloma.

In one embodiment, the multiple myeloma is newly diagnosed multiple myeloma (NDMM). In one embodiment, the subject is transplant-eligible. In one embodiment, the subject is eligible for autologous stem cell transplant (ASCT).

In one embodiment, a compound provided herein (e.g., Compound 1, or a pharmaceutically acceptable salt there of (e.g., a hydrobromide salt)) is administered in combination with a second therapeutic agent provided herein as a first line treatment of the multiple myeloma.

In one embodiment, a compound provided herein (e.g., Compound 1, or a pharmaceutically acceptable salt there of (e.g., a hydrobromide salt)) is administered in combination with a second therapeutic agent provided herein as an induction therapy. In one embodiment, the induction therapy is followed by an autologous stem cell transplant (ASCT) with or without maintenance as part of standard of care (SOC).

In one embodiment, the multiple myeloma is relapsed or refractory multiple myeloma (RRMM). In one embodiment, the subject has received at least one prior line of therapy. In one embodiment, the subject has received at least two prior lines of therapy. In one embodiment, the subject has received one to three prior lines of therapy. In one embodiment, the subject has received two to four prior lines of therapy. In one embodiment, the prior line of therapy is anti-myeloma therapy. In one embodiment, the subject achieved a response (minimal response [MR] or better) to at least 1 prior line of therapy. In one embodiment, the subject has disease progression (e.g., within 6 months) after achieving at least a partial response to the prior line of therapy. In one embodiment, the prior lines of therapy include a lenalidomide-containing therapy. In one embodiment, the prior lines of therapy include a proteasome inhibitor. In one embodiment, the proteasome inhibitor is bortezomib. In one embodiment, the proteasome inhibitor is carfilzomib. In one embodiment, the proteasome inhibitor is ixazomib.

In one embodiment, also provided herein are methods for inducing a therapeutic response assessed with the International Uniform Response Criteria for Multiple Myeloma (IURC) (see Durie B G M, Harousseau J-L, Miguel J S, et al. International uniform response criteria for multiple myeloma. Leukemia, 2006; (10) 10: 1-7) of a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving a stringent complete response, complete response, or very good partial response, as determined by the International Uniform Response Criteria for Multiple Myeloma (IURC) in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving an increase in overall survival, progression-free survival, event-free survival, time to progression, or disease-free survival in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving an increase in overall survival in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving an increase in progression-free survival in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving an increase in event-free survival in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving an increase in time to progression in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

In another embodiment, provided herein are methods for achieving an increase in disease-free survival in a patient, comprising administering to a patient having multiple myeloma an effective amount of a compound provided herein (e.g., Compound 1, or a pharmaceutically acceptable salt there of (e.g., a hydrobromide salt)), in combination with a second active agent provided herein.

Also provided herein are methods of treating patients who have been previously treated for multiple myeloma but are non-responsive to standard therapies, as well as those who have not previously been treated. Further encompassed are methods of treating patients who have undergone surgery in an attempt to treat multiple myeloma, as well as those who have not. Also provided herein are methods of treating patients who have been previously undergone transplant therapy, as well as those who have not.

The methods provided herein include treatment of multiple myeloma that is relapsed, refractory or resistant. The methods provided herein include prevention of multiple myeloma that is relapsed, refractory or resistant. The methods provided herein include management of multiple myeloma that is relapsed, refractory or resistant. In some such embodiments, the myeloma is primary, secondary, tertiary, quadruply or quintuply relapsed multiple myeloma. In one embodiment, the methods provided herein reduce, maintain or eliminate minimal residual disease (MRD). In one embodiment, provided herein is a method of increasing rate and/or durability of MRD negativity in multiple myeloma patients, comprising administering an effective amount of a compound provided herein (e.g., Compound 1, or a pharmaceutically acceptable salt there of (e.g., a hydrobromide salt)), in combination with a second active agent provided herein. In one embodiment, methods provided herein encompass treating, preventing or managing various types of multiple myeloma, such as monoclonal gammopathy of undetermined significance (MGUS), low risk, intermediate risk, and high risk multiple myeloma, newly diagnosed multiple myeloma (including low risk, intermediate risk, and high risk newly diagnosed multiple myeloma), transplant eligible and transplant ineligible multiple myeloma, smoldering (indolent) multiple myeloma (including low risk, intermediate risk, and high risk smouldering multiple myeloma), active multiple myeloma, solitary plasmacytoma, extramedullary plasmacytoma, plasma cell leukemia, central nervous system multiple myeloma, light chain myeloma, non-secretory myeloma, Immunoglobulin D myeloma, and Immunoglobulin E myeloma, by administering a therapeutically effective amount of a compound described herein. In another embodiment, methods provided herein encompass treating, preventing or managing multiple myeloma characterized by genetic abnormalities, such as Cyclin D translocations (for example, t(11; 14)(q13;q32); t(6; 14)(p21;32); t(12;14)(p13;q32); or t(6;20);); MMSET translocations (for example, t(4;14)(p16;q32)); MAF translocations (for example, t(14;16)(q32;q32); t(20;22); t(16; 22)(q11;q13); or t(14;20)(q32;q11)); or other chromosome factors (for example, deletion of 17p13, or chromosome 13; del(17/17p), nonhyperdiploidy, and gain(1q)), by administering a therapeutically effective amount of a compound described herein. In one embodiment, the multiple myeloma is characterized according to the multiple myeloma International Staging System (ISS). In one embodiment, the multiple myeloma is Stage I multiple myeloma as characterized by ISS (e.g., serum β2 microglobulin <3.5 mg/L and serum albumin ≥3.5 g/dL). In one embodiment, the multiple myeloma is Stage III multiple myeloma as characterized by ISS (e.g., serum β2 microglobulin >5.4 mg/L). In one embodiment, the multiple myeloma is Stage II multiple myeloma as characterized by ISS (e.g., not Stage I or III).

In some embodiments, the methods comprise administering a therapeutically effective amount of Compound 1, or an enantiomer, mixture of enantiomers, tautomer, isotopolog, or pharmaceutically acceptable salt thereof, in combination with a second active agent provided herein as induction therapy. In some embodiments, the methods comprise administering a therapeutically effective amount of Compound 1, or an enantiomer, mixture of enantiomers, tautomer, isotopolog, or pharmaceutically acceptable salt thereof, in combination with a second active agent provided herein as consolidation therapy. In some embodiments, the methods comprise administering a therapeutically effective amount of Compound 1, or an enantiomer, mixture of enantiomers, tautomer, isotopolog, or pharmaceutically acceptable salt thereof, in combination with a second active agent provided herein as maintenance therapy.

In one embodiment of the methods described herein, the multiple myeloma is high risk multiple myeloma. In some such embodiments, the high risk multiple myeloma is relapsed or refractory. In one embodiment, the high risk multiple myeloma is multiple myeloma that is relapsed within 12 months of first treatment. In yet another embodiment, the high risk multiple myeloma is multiple myeloma that is characterized by genetic abnormalities, for example, one or more of del(17/17p) and t(14;16)(q32;q32). In some such embodiments, the high risk multiple myeloma is relapsed or refractory to one, two or three previous treatments.

In one embodiment, the multiple myeloma is characterized by a p53 mutation. In one embodiment, the p53 mutation is a Q331 mutation. In one embodiment, the p53 mutation is an R273H mutation. In one embodiment, the p53 mutation is a K132 mutation. In one embodiment, the p53 mutation is a K132N mutation. In one embodiment, the p53 mutation is an R337 mutation. In one embodiment, the p53 mutation is an R337L mutation. In one embodiment, the p53 mutation is a W146 mutation. In one embodiment, the p53 mutation is an S261 mutation. In one embodiment, the p53 mutation is an S261T mutation. In one embodiment, the p53 mutation is an E286 mutation. In one embodiment, the p53 mutation is an E286K mutation. In one embodiment, the p53 mutation is an R175 mutation. In one embodiment, the p53 mutation is an R175H mutation. In one embodiment, the p53 mutation is an E258 mutation. In one embodiment, the p53 mutation is an E258K mutation. In one embodiment, the p53 mutation is an A161 mutation. In one embodiment, the p53 mutation is an A161T mutation.

In one embodiment, the multiple myeloma is characterized by homozygous deletion of p53. In one embodiment, the multiple myeloma is characterized by homozygous deletion of wild type p53.

In one embodiment, the multiple myeloma is characterized by wild type p53.

In one embodiment, the multiple myeloma is characterized by activation of one or more oncogenic drivers. In one embodiment, the one or more oncogenic drivers are selected from the group consisting of C-MAF, MAFB, FGFR3, MMset, Cyclin D1, and Cyclin D. In one embodiment, the multiple myeloma is characterized by activation of C-MAF. In one embodiment, the multiple myeloma is characterized by activation of MAFB. In one embodiment, the multiple myeloma is characterized by activation of FGFR3 and MMset. In one embodiment, the multiple myeloma is characterized by activation of C-MAF, FGFR3, and MMset. In one embodiment, the multiple myeloma is characterized by activation of Cyclin D1. In one embodiment, the multiple myeloma is characterized by activation of MAFB and Cyclin D1. In one embodiment, the multiple myeloma is characterized by activation of Cyclin D.

In one embodiment, the multiple myeloma is characterized by one or more chromosomal translocations. In one embodiment, the chromosomal translocation is t(14;16). In one embodiment, the chromosomal translocation is t(14;20). In one embodiment, the chromosomal translocation is t(4;14). In one embodiment, the chromosomal translocations are t(4;14) and t(14;16). In one embodiment, the chromosomal translocation is t(11;14). In one embodiment, the chromosomal translocation is t(6;20). In one embodiment, the chromosomal translocation is t(20;22). In one embodiment, the chromosomal translocations are t(6;20) and t(20;22). In one embodiment, the chromosomal translocation is t(16;22). In one embodiment, the chromosomal translocations are t(14;16) and t(16;22). In one embodiment, the chromosomal translocations are t(14;20) and t(11;14).

In one embodiment, the multiple myeloma is characterized by a Q331 p53 mutation, by activation of C-MAF, and by a chromosomal translocation at t(14;16). In one embodiment, the multiple myeloma is characterized by homozygous deletion of p53, by activation of C-MAF, and by a chromosomal translocation at t(14;16). In one embodiment, the multiple myeloma is characterized by a K132N p53 mutation, by activation of MAFB, and by a chromosomal translocation at t(14;20). In one embodiment, the multiple myeloma is characterized by wild type p53, by activation of FGFR3 and MMset, and by a chromosomal translocation at t(4;14). In one embodiment, the multiple myeloma is characterized by wild type p53, by activation of C-MAF, and by a chromosomal translocation at t(14;16). In one embodiment, the multiple myeloma is characterized by homozygous deletion of p53, by activation of FGFR3, MMset, and C-MAF, and by chromosomal translocations at t(4;14) and t(14;16). In one embodiment, the multiple myeloma is characterized by homozygous deletion of p53, by activation of Cyclin D1, and by a chromosomal translocation at t(11;14). In one embodiment, the multiple myeloma is characterized by an R337L p53 mutation, by activation of Cyclin D1, and by a chromosomal translocation at t(11;14). In one embodiment, the multiple myeloma is characterized by a W146 p53 mutation, by activation of FGFR3 and MMset, and by a chromosomal translocation at t(4;14). In one embodiment, the multiple myeloma is characterized by an S261T p53 mutation, by activation of MAFB, and by chromosomal translocations at t(6;20) and t(20;22). In one embodiment, the multiple myeloma is characterized by an E286K p53 mutation, by activation of FGFR3 and MMset, and by a chromosomal translocation at t(4;14). In one embodiment, the multiple myeloma is characterized by an R175H p53 mutation, by activation of FGFR3 and MMset, and by a chromosomal translocation at t(4;14). In one embodiment, the multiple myeloma is characterized by an E258K p53 mutation, by activation of C-MAF, and by chromosomal translocations at t(14;16) and t(16;22). In one embodiment, the multiple myeloma is characterized by wild type p53, by activation of MAFB and Cyclin D1, and by chromosomal translocations at t(14;20) and t(11;14). In one embodiment, the multiple myeloma is characterized by an A161T p53 mutation, by activation of Cyclin D, and by a chromosomal translocation at t(11;14).

In some embodiments of the methods described herein, the multiple myeloma is transplant eligible newly diagnosed multiple myeloma. In another embodiment, the multiple myeloma is transplant ineligible newly diagnosed multiple myeloma.

In yet other embodiments, the multiple myeloma is characterized by early progression (for example less than 12 months) following initial treatment. In still other embodiments, the multiple myeloma is characterized by early progression (for example less than 12 months) following autologous stem cell transplant. In another embodiment, the multiple myeloma is refractory to lenalidomide. In another embodiment, the multiple myeloma is refractory to pomalidomide. In some such embodiments, the multiple myeloma is predicted to be refractory to pomalidomide (for example, by molecular characterization). In another embodiment, the multiple myeloma is relapsed or refractory to 3 or more treatments and was exposed to a proteasome inhibitor (for example, bortezomib, carfilzomib, ixazomib, oprozomib, or marizomib) and an immunomodulatory compound (for example thalidomide, lenalidomide, pomalidomide, iberdomide, or avadomide), or double refractory to a proteasome inhibitor and an immunomodulatory compound. In still other embodiments, the multiple myeloma is relapsed or refractory to 3 or more prior therapies, including for example, a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide) or double refractory to a proteasome inhibitor or immunomodulatory compound and a CD38 mAb. In still other embodiments, the multiple myeloma is triple refractory, for example, the multiple myeloma is refractory to a proteasome inhibitor (for example, bortezomib, carfilzomib, ixazomib, oprozomib or marizomib), an immunomodulatory compound (for example thalidomide, lenalidomide, pomalidomide, iberdomide, or avadomide), and one other active agent, as described herein.

In certain embodiments, provided herein are methods of treating, preventing, and/or managing multiple myeloma, including relapsed/refractory multiple myeloma in patients with impaired renal function or a symptom thereof, comprising administering a therapeutically effective amount of a compound provided herein (e.g., Compound 1, or a pharmaceutically acceptable salt thereof), in combination with a second active agent provided herein, to a patient having relapsed/refractory multiple myeloma with impaired renal function.

In certain embodiments, provided herein are methods of treating, preventing, and/or managing multiple myeloma, including relapsed or refractory multiple myeloma in frail patients or a symptom thereof, comprising administering a therapeutically effective amount of a compound provided herein (e.g., Compound 1, or a pharmaceutically acceptable salt thereof), in combination with a second active agent provided herein, to a frail patient having multiple myeloma. In some such embodiments, the frail patient is characterized by ineligibility for induction therapy, or intolerance to dexamethasone treatment. In some such embodiment the frail patient is elderly, for example, older than 65 years old.

In certain embodiments, provided herein are methods of treating, preventing or managing multiple myeloma, comprising administering to a patient a therapeutically effective amount of a compound provided herein (e.g., Compound 1, or a pharmaceutically acceptable salt thereof), in combination with a second active agent provided herein, wherein the multiple myeloma is fourth line relapsed/refractory multiple myeloma.

In certain embodiments, provided herein are methods of treating, preventing or managing multiple myeloma, comprising administering to a patient a therapeutically effective amount of a compound provided herein (e.g., Compound 1, or a pharmaceutically acceptable salt thereof), in combination with a second active agent provided herein, as induction therapy, wherein the multiple myeloma is newly diagnosed, transplant-eligible multiple myeloma.

In certain embodiments, provided herein are methods of treating, preventing or managing multiple myeloma, comprising administering to a patient a therapeutically effective amount of a compound provided herein (e.g., Compound 1, or a pharmaceutically acceptable salt thereof), in combination with a second active agent provided herein, as maintenance therapy after other therapy or transplant, wherein the multiple myeloma is newly diagnosed, transplant-eligible multiple myeloma prior to the other therapy or transplant.

In certain embodiments, provided herein are methods of treating, preventing or managing multiple myeloma, comprising administering to a patient a therapeutically effective amount of a compound provided herein (e.g., Compound 1, or a pharmaceutically acceptable salt thereof), in combination with a second active agent provided herein, as maintenance therapy after other therapy or transplant. In some embodiments, the multiple myeloma is newly diagnosed, transplant-eligible multiple myeloma prior to the other therapy and/or transplant. In some embodiments, the other therapy prior to transplant is treatment with chemotherapy or a compound provided herein (e.g., Compound 1, or a pharmaceutically acceptable salt thereof).

In certain embodiments, provided herein are methods of treating, preventing or managing multiple myeloma, comprising administering to a patient a therapeutically effective amount of a compound provided herein (e.g., Compound 1, or a pharmaceutically acceptable salt thereof), in combination with a second active agent provided herein, wherein the multiple myeloma is high risk multiple myeloma, that is relapsed or refractory to one, two or three previous treatments.

In certain embodiments, provided herein are methods of treating, preventing or managing multiple myeloma, comprising administering to a patient a therapeutically effective amount of a compound provided herein (e.g., Compound 1, or a pharmaceutically acceptable salt thereof), in combination with a second active agent provided herein, wherein the multiple myeloma is newly diagnosed, transplant-ineligible multiple myeloma.

In certain embodiments, the patient to be treated with one of the methods provided herein has not been treated with multiple myeloma therapy prior to the administration of a compound provided herein (e.g., Compound 1, or a pharmaceutically acceptable salt thereof), in combination with a second active agent provided herein. In certain embodiments, the patient to be treated with one of the methods provided herein has developed drug resistance to the anti-multiple myeloma therapy. In some such embodiments, the patient has developed resistance to one, two, or three anti-multiple myeloma therapies, wherein the therapies are selected from a CD38 monoclonal antibody (CD38 mAb, for example, daratumumab or isatuximab), a proteasome inhibitor (for example, bortezomib, carfilzomib, ixazomib, or marizomib), and an immunomodulatory compound (for example thalidomide, lenalidomide, pomalidomide, iberdomide, or avadomide).

The methods provided herein encompass treating a patient regardless of patient's age. In some embodiments, the subject is 18 years or older. In other embodiments, the subject is more than 18, 25, 35, 40, 45, 50, 55, 60, 65, or 70 years old. In other embodiments, the subject is less than 65 years old. In other embodiments, the subject is more than 65 years old. In one embodiment, the subject is an elderly multiple myeloma subject, such as a subject older than 65 years old. In one embodiment, the subject is an elderly multiple myeloma subject, such as a subject older than 75 years old.

In one embodiment, the second therapeutic agent is a combination of elotuzumab and dexamethasone.

In one embodiment, provided herein is a method of treating multiple myeloma, comprising administering to a subject in need thereof a therapeutically effective amount of a compound of Formula (I):

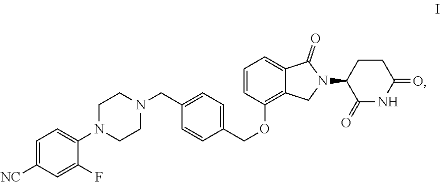 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with elotuzumab and dexamethasone. In one embodiment, provided herein is a compound for use in a method of treating multiple myeloma, wherein the method comprises administering to a subject in need thereof a therapeutically effective amount of the compound characterized by Formula (I), or an enantiomer, mixture of enantiomers, tautomer, isotopolog, or pharmaceutically acceptable salt thereof, in combination with elotuzumab and dexamethasone.

Elotuzumab is a humanized IgG1 monoclonal antibody that specifically targets the Signaling Lymphocytic Activation Molecule Family member 7 (SLAMF7) protein. In one embodiment, elotuzumab is administered in an amount according to the physician's decision. In one embodiment, elotuzumab is administered according to the locally approved label or pharmacy manual for preparation, administration, and storage information. In one embodiment, elotuzumab is administered according to the label of Empliciti®. In one embodiment, elotuzumab is administered at a dose of about 10 mg/kg per day. In one embodiment, elotuzumab is administered at a dose of about 20 mg/kg per day. In one embodiment, elotuzumab is administered on days 1, 8, 15, and 22 of first two 28-day cycles, and on day 1 of subsequent 28-day cycle(s). In one embodiment, elotuzumab is administered intravenously. In one embodiment, elotuzumab is administered via intravenous injection. In one embodiment, elotuzumab is administered via intravenous infusion.

In one embodiment, dexamethasone is administered in an amount according to the physician's decision. In one embodiment, dexamethasone is administered according to the locally approved label or pharmacy manual for preparation, administration, and storage information. In one embodiment, dexamethasone is administered at a dose of about 40 mg per day. In one embodiment, dexamethasone is administered at a dose of about 36 mg per day. In one embodiment, dexamethasone is administered at a dose of about 28 mg per day. In one embodiment, dexamethasone is administered at a dose of about 20 mg per day. In one embodiment, dexamethasone is administered at a dose of about 16 mg per day. In one embodiment, dexamethasone is administered at a dose of about 8 mg per day. In one embodiment, dexamethasone is administered on days 1, 8, 15, and 22 of a 28-day cycle. In one embodiment, dexamethasone is administered orally. In one embodiment, dexamethasone is administered intravenously. In one embodiment, dexamethasone is administered via intravenous injection. In one embodiment, dexamethasone is administered via intravenous infusion.

In one embodiment, elotuzumab is administered on days 1, 8, 15, and 22 of first two 28-day cycles, and on day 1 of subsequent 28-day cycle(s); and dexamethasone is administered on days 1, 8, 15, and 22 of each of the 28-day cycles.

In one embodiment, elotuzumab is administered intravenously, and dexamethasone is administered intravenously or orally.

In one embodiment, elotuzumab is administered intravenously at a dose of about 10 mg/kg on days 1, 8, 15, and 22 of first two 28-day cycles, and at a dose of about 20 mg/kg on day 1 of subsequent 28-day cycle(s); and dexamethasone is administered both orally at a dose of about 28 mg and intravenously at a dose of about 8 mg (for a total of 36 mg) on days 1, 8, 15, and 22 of first two 28-day cycles and on day 1 of subsequent 28-day cycle(s) starting on the third 28-day cycle, and is administered orally at a dose of about 40 mg on days 8, 15, and 22 of subsequent 28-day cycle(s) starting on the third 28-day cycle.

In one embodiment, elotuzumab is administered intravenously at a dose of about 10 mg/kg on days 1, 8, 15, and 22 of first two 28-day cycles, and at a dose of about 20 mg/kg on day 1 of subsequent 28-day cycle(s); and dexamethasone is administered both orally at a dose of about 8 mg and intravenously at a dose of about 8 mg (for a total of 16 mg) on days 1, 8, 15, and 22 of first two 28-day cycles and on day 1 of subsequent 28-day cycle(s) starting on the third 28-day cycle, and is administered orally at a dose of about 20 mg on days 8, 15, and 22 of subsequent 28-day cycle(s) starting on the third 28-day cycle.

In one embodiment, the second therapeutic agent is a combination of isatuximab and dexamethasone.

In one embodiment, provided herein is a method of treating multiple myeloma, comprising administering to a subject in need thereof a therapeutically effective amount of a compound of Formula (I):

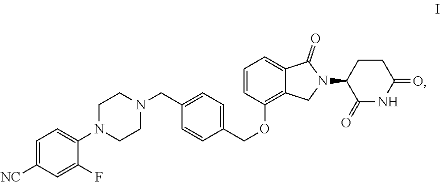 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with isatuximab and dexamethasone. In one embodiment, provided herein is a compound for use in a method of treating multiple myeloma, wherein the method comprises administering to a subject in need thereof a therapeutically effective amount of the compound characterized by Formula (I), or an enantiomer, mixture of enantiomers, tautomer, isotopolog, or pharmaceutically acceptable salt thereof, in combination with isatuximab and dexamethasone.

Isatuximab is an immunoglobulin G1 (IgG1)-derived monoclonal antibody that binds to CD38 expressed on the surface of hematopoietic and tumor cells, including MM cells. In one embodiment, isatuximab is administered in an amount according to the physician's decision. In one embodiment, isatuximab is administered according to the locally approved label or pharmacy manual for preparation, administration, and storage information. In one embodiment, isatuximab is administered according to the label of Sarclisa®. In one embodiment, isatuximab is administered at a dose of about 10 mg/kg per day. In one embodiment, isatuximab is administered on days 1, 8, 15, and 22 of a first 28-day cycle, and on days 1 and 15 of subsequent 28-day cycle(s). In one embodiment, isatuximab is administered intravenously. In one embodiment, isatuximab is administered via intravenous injection. In one embodiment, isatuximab is administered via intravenous infusion.

In one embodiment, dexamethasone is administered in an amount according to the physician's decision. In one embodiment, dexamethasone is administered according to the locally approved label or pharmacy manual for preparation, administration, and storage information. In one embodiment, dexamethasone is administered at a dose of about 40 mg per day. In one embodiment, dexamethasone is administered at a dose of about 20 mg per day. In one embodiment, dexamethasone is administered on days 1, 8, 15, and 22 of a 28-day cycle. In one embodiment, dexamethasone is administered orally. In one embodiment, dexamethasone is administered intravenously. In one embodiment, dexamethasone is administered via intravenous injection. In one embodiment, dexamethasone is administered via intravenous infusion.

In one embodiment, isatuximab is administered on days 1, 8, 15, and 22 of a first 28-day cycle, and on days 1 and 15 of subsequent 28-day cycle(s); and dexamethasone is administered on days 1, 8, 15, and 22 of each of the 28-day cycles.

In one embodiment, isatuximab is administered intravenously, and dexamethasone is administered intravenously or orally.

In one embodiment, isatuximab is administered intravenously at a dose of about 10 mg/kg on days 1, 8, 15, and 22 of a first 28-day cycle, and at a dose of about 10 mg/kg on days 1 and 15 of subsequent 28-day cycle(s); and dexamethasone is administered intravenously or orally at a dose of about 40 mg on days 1, 8, 15, and 22 of each of the 28-day cycles.

In one embodiment, isatuximab is administered intravenously at a dose of about 10 mg/kg on days 1, 8, 15, and 22 of a first 28-day cycle, and at a dose of about 10 mg/kg on days 1 and 15 of subsequent 28-day cycle(s); and dexamethasone is administered intravenously or orally at a dose of about 20 mg on days 1, 8, 15, and 22 of each of the 28-day cycles.

In one embodiment, the second therapeutic agent is a combination of bortezomib and dexamethasone.

In one embodiment, provided herein is a method of treating multiple myeloma, comprising administering to a subject in need thereof a therapeutically effective amount of a compound of Formula (I):

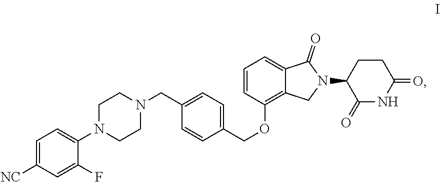 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with bortezomib and dexamethasone.

Bortezomib is a proteasome inhibitor. In one embodiment, bortezomib is administered in an amount according to the physician's decision. In one embodiment, bortezomib is administered according to the locally approved label or pharmacy manual for preparation, administration, and storage information. In one embodiment, bortezomib is administered according to the label of Velcade®. In one embodiment, bortezomib is administered at a dose of about 1.3 mg/m² per day. In one embodiment, bortezomib is administered on days 1, 4, 8 and 11 of first eight 21-day cycles, and on days 1 and 8 of subsequent 21-day cycle(s). In one embodiment, bortezomib is administered on days 1, 4, 8 and 11 of up to six 21-day cycles. In one embodiment, bortezomib is administered on days 1, 4, 8 and 11 of six 21-day cycles. In one embodiment, bortezomib is administered subcutaneously. In one embodiment, bortezomib is administered via subcutaneous infusion.

In one embodiment, dexamethasone is administered in an amount according to the physician's decision. In one embodiment, dexamethasone is administered according to the locally approved label or pharmacy manual for preparation, administration, and storage information. In one embodiment, dexamethasone is administered at a dose of about 20 mg per day. In one embodiment, dexamethasone is administered at a dose of about 10 mg per day. In one embodiment, dexamethasone is administered on days 1, 2, 4, 5, 8, 9, 11 and 12 of first eight 21-day cycles, and on days 1, 2, 8 and 9 of subsequent 21-day cycle(s). In one embodiment, dexamethasone is administered on days 1, 2, 4, 5, 8, 9, 11 and 12 of up to six 21-day cycles. In one embodiment, dexamethasone is administered on days 1, 2, 4, 5, 8, 9, 11 and 12 of six 21-day cycles. In one embodiment, dexamethasone is administered orally.

In one embodiment, bortezomib is administered on days 1, 4, 8 and 11 of first eight 21-day cycles, and on days 1 and 8 of subsequent 21-day cycle(s); and dexamethasone is administered on days 1, 2, 4, 5, 8, 9, 11 and 12 of first eight 21-day cycles, and on days 1, 2, 8 and 9 of subsequent 21-day cycle(s).

In one embodiment, bortezomib is administered on days 1, 4, 8 and 11 of (up to) six 21-day cycles; and dexamethasone is administered on days 1, 2, 4, 5, 8, 9, 11 and 12 of each of the 21-day cycles.

In one embodiment, bortezomib is administered subcutaneously, and dexamethasone is administered orally.

In one embodiment, bortezomib is administered subcutaneously at a dose of about 1.3 mg/m² on days 1, 4, 8 and 11 of first eight 21-day cycles, and on days 1 and 8 of subsequent 21-day cycle(s); and dexamethasone is administered orally at a dose of about 20 mg on days 1, 2, 4, 5, 8, 9, 11 and 12 of first eight 21-day cycles, and on days 1, 2, 8 and 9 of subsequent 21-day cycle(s).

In one embodiment, bortezomib is administered subcutaneously at a dose of about 1.3 mg/m² on days 1, 4, 8 and 11 of first eight 21-day cycles, and on days 1 and 8 of subsequent 21-day cycle(s); and dexamethasone is administered orally at a dose of about 10 mg on days 1, 2, 4, 5, 8, 9, 11 and 12 of first eight 21-day cycles, and on days 1, 2, 8 and 9 of subsequent 21-day cycle(s).

In one embodiment, bortezomib is administered subcutaneously at a dose of about 1.3 mg/m² on days 1, 4, 8 and 11 of (up to) six 21-day cycles; and dexamethasone is administered orally at a dose of about 20 mg on days 1, 2, 4, 5, 8, 9, 11 and 12 of each of the 21-day cycles.

In one embodiment, bortezomib is administered subcutaneously at a dose of about 1.3 mg/m² on days 1, 4, 8 and 11 of (up to) six 21-day cycles; and dexamethasone is administered orally at a dose of about 10 mg on days 1, 2, 4, 5, 8, 9, 11 and 12 of each of the 21-day cycles.

In one embodiment, the second therapeutic agent is a combination of daratumumab and dexamethasone.

In one embodiment, provided herein is a method of treating multiple myeloma, comprising administering to a subject in need thereof a therapeutically effective amount of a compound of Formula (I):

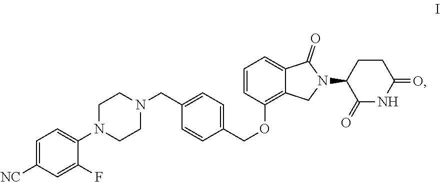 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with daratumumab and dexamethasone.

Daratumumab is a human immunoglobulin G (IgG)κ monoclonal antibody that targets CD38, a cell-surface molecule that is expressed by malignant plasma cells. In one embodiment, daratumumab is administered in an amount according to the physician's decision. In one embodiment, daratumumab is administered according to the locally approved label or pharmacy manual for preparation, administration, and storage information. In one embodiment, daratumumab is administered according to the label of Darzalex®. In one embodiment, daratumumab is administered at a dose of about 16 mg/kg per day. In one embodiment, daratumumab is administered at a dose of about 1800 mg per day. In one embodiment, daratumumab is administered on days 1, 8, 15, and 22 of first two 28-day cycles, on days 1 and 15 of the third to sixth 28-day cycles, and on day 1 of subsequent 28-day cycle(s). In one embodiment, daratumumab is administered on days 1, 8, and 15 of first three 21-day cycles, on day 1 of the fourth to eighth 21-day cycles, and on day 1 of subsequent 28-day cycle(s). In one embodiment, daratumumab is administered intravenously. In one embodiment, daratumumab is administered via intravenous injection. In one embodiment, daratumumab is administered via intravenous infusion. In one embodiment, daratumumab is administered subcutaneously. In one embodiment, daratumumab is administered via subcutaneous infusion.

In one embodiment, dexamethasone is administered in an amount according to the physician's decision. In one embodiment, dexamethasone is administered according to the locally approved label or pharmacy manual for preparation, administration, and storage information. In one embodiment, dexamethasone is administered at a dose of about 40 mg per day. In one embodiment, dexamethasone is administered at a dose of about 20 mg per day. In one embodiment, dexamethasone is administered on days 1, 8, 15, and 22 of a 28-day cycle. In one embodiment, dexamethasone is administered on days 1, 8, and 15 of first eight 21-day cycles, and on days 1, 8, 15, and 22 of subsequent 28-day cycle(s). In one embodiment, dexamethasone is administered orally. In one embodiment, dexamethasone is administered intravenously. In one embodiment, dexamethasone is administered via intravenous injection. In one embodiment, dexamethasone is administered via intravenous infusion.

In one embodiment, daratumumab is administered on days 1, 8, 15, and 22 of first two 28-day cycles, on days 1 and 15 of the third to sixth 28-day cycles, and on day 1 of subsequent 28-day cycle(s); and dexamethasone is administered on days 1, 8, 15, and 22 of each of the 28-day cycles.

In one embodiment, daratumumab is administered on days 1, 8, and 15 of first three 21-day cycles, on day 1 of the fourth to eighth 21-day cycles, and on day 1 of subsequent 28-day cycle(s); and dexamethasone is administered on days 1, 8, and 15 of first eight 21-day cycles, and on days 1, 8, 15, and 22 of subsequent 28-day cycle(s).

In one embodiment, daratumumab is administered intravenously or subcutaneously, and dexamethasone is administered intravenously or orally.

In one embodiment, daratumumab is administered intravenously at a dose of about 16 mg/kg or subcutaneously at a dose of about 1800 mg on days 1, 8, 15, and 22 of first two 28-day cycles, on days 1 and 15 of the third to sixth 28-day cycles, and on day 1 of subsequent 28-day cycle(s); and dexamethasone is administered orally or intravenously at a dose of about 40 mg on days 1, 8, 15, and 22 of each of the 28-day cycles.

In one embodiment, daratumumab is administered intravenously at a dose of about 16 mg/kg or subcutaneously at a dose of about 1800 mg on days 1, 8, 15, and 22 of first two 28-day cycles, on days 1 and 15 of the third to sixth 28-day cycles, and on day 1 of subsequent 28-day cycle(s); and dexamethasone is administered orally or intravenously at a dose of about 20 mg on days 1, 8, 15, and 22 of each of the 28-day cycles.

In one embodiment, daratumumab is administered intravenously at a dose of about 16 mg/kg or subcutaneously at a dose of about 1800 mg on days 1, 8, and 15 of first three 21-day cycles, on day 1 of the fourth to eighth 21-day cycles, and on day 1 of subsequent 28-day cycle(s); and dexamethasone is administered orally or intravenously at a dose of about 40 mg on days 1, 8, and 15 of first eight 21-day cycles, and on days 1, 8, 15, and 22 of subsequent 28-day cycle(s).

In one embodiment, daratumumab is administered intravenously at a dose of about 16 mg/kg or subcutaneously at a dose of about 1800 mg on days 1, 8, and 15 of first three 21-day cycles, on day 1 of the fourth to eighth 21-day cycles, and on day 1 of subsequent 28-day cycle(s); and dexamethasone is administered orally or intravenously at a dose of about 20 mg on days 1, 8, and 15 of first eight 21-day cycles, and on days 1, 8, 15, and 22 of subsequent 28-day cycle(s).

In one embodiment, the second therapeutic agent is a combination of carfilzomib and dexamethasone.

In one embodiment, provided herein is a method of treating multiple myeloma, comprising administering to a subject in need thereof a therapeutically effective amount of a compound of Formula (I):

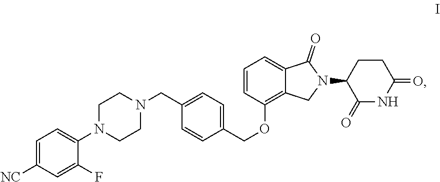 [SMILES: N#Cc1ccc(N2CCN(Cc3ccc(COc4cccc5c4CN([C@H]4CCC(=O)NC4=O)C5=O)cc3)CC2)c(F)c1]

or an enantiomer, mixture of enantiomers, tautomer, isotopolog, or pharmaceutically acceptable salt thereof, in combination with carfilzomib and dexamethasone.

Carfilzomib is a tetrapeptide epoxyketone proteasome inhibitor that irreversibly binds to the N-terminal threonine-containing active sites of the 20S proteasome. In one embodiment, carfilzomib is administered in an amount according to the physician's decision. In one embodiment, carfilzomib is administered according to the locally approved label or pharmacy manual for preparation, administration, and storage information. In one embodiment, carfilzomib is administered according to the label of Kyprolis®. In one embodiment, carfilzomib is administered at a dose of about 20 mg/m² per day. In one embodiment, carfilzomib is administered at a dose of about 56 mg/m² per day. In one embodiment, carfilzomib is administered on days 1, 8, and 15 of first twelve 28-day cycles, and on days 1 and 15 of subsequent 28-day cycle(s). In one embodiment, carfilzomib is administered intravenously. In one embodiment, carfilzomib is administered via intravenous injection. In one embodiment, carfilzomib is administered via intravenous infusion.

In one embodiment, dexamethasone is administered in an amount according to the physician's decision. In one embodiment, dexamethasone is administered according to the locally approved label or pharmacy manual for preparation, administration, and storage information. In one embodiment, dexamethasone is administered at a dose of about 40 mg per day. In one embodiment, dexamethasone is administered at a dose of about 20 mg per day. In one embodiment, dexamethasone is administered on days 1, 8, 15, and 22 of a 28-day cycle. In one embodiment, dexamethasone is administered orally. In one embodiment, dexamethasone is administered intravenously. In one embodiment, dexamethasone is administered via intravenous injection. In one embodiment, dexamethasone is administered via intravenous infusion.

In one embodiment, carfilzomib is administered on days 1, 8, and 15 of first twelve 28-day cycles, and on days 1 and 15 of subsequent 28-day cycle(s); and dexamethasone is administered on days 1, 8, 15, and 22 of each of the 28-day cycles.

In one embodiment, carfilzomib is administered intravenously, and dexamethasone is administered intravenously or orally.

In one embodiment, carfilzomib is administered intravenously at a dose of about 20 mg/m² on day 1 of first 28-day cycle, at a dose of about 56 mg/m² on days 8 and 15 of first 28-day cycle, at a dose of about 56 mg/m² on days 1, 8, and 15 of the second to twelfth 28-day cycles, and at a dose of about 56 mg/m² on days 1 and 15 of subsequent 28-day cycle(s); and dexamethasone is administered intravenously or orally at a dose of about 40 mg on days 1, 8, 15, and 22 of each of the 28-day cycles.

In one embodiment, carfilzomib is administered intravenously at a dose of about 20 mg/m² on day 1 of first 28-day cycle, at a dose of about 56 mg/m² on days 8 and 15 of first 28-day cycle, at a dose of about 56 mg/m² on days 1, 8, and 15 of the second to twelfth 28-day cycles, and at a dose of about 56 mg/m² on days 1 and 15 of subsequent 28-day cycle(s); and dexamethasone is administered intravenously or orally at a dose of about 20 mg on days 1, 8, 15, and 22 of each of the 28-day cycles.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before) to the administration of a second therapeutic agent provided herein to the subject.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered concomitantly with the administration of a second therapy provided herein to the subject.

In one embodiment, a first therapy (e.g., a prophylactic or therapeutic agent such as Compound 1, or an enantiomer, mixture of enantiomers, tautomer, isotopolog, or pharmaceutically acceptable salt thereof) provided herein is administered subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent provided herein to the subject.

In one embodiment, a compound of Formula (I), or a pharmaceutically acceptable salt thereof, is administered. In one embodiment, a compound of Formula (I) (free base) is administered. In one embodiment, a hydrobromide salt of a compound of Formula (I) is administered. In one embodiment, the compound is administered orally.

In one embodiment, the compound is administered once daily (QD), or divided into multiple daily doses such as twice daily (BID), three times daily (TID), and four times daily (QID). In addition, the administration can be continuous (i.e., daily for consecutive days or every day), intermittent, e.g., in cycles (i.e., including days, weeks, or months of rest without drug).

In one embodiment, a compound described herein, e.g., Compound 1, or pharmaceutically acceptable salt thereof (e.g., a hydrobromide salt of Compound 1), is administered at a dose of from about 0.1 mg to about 2 mg once daily. In one embodiment, the compound is administered at a dose of from about 0.3 mg to about 0.6 mg once daily. In one embodiment, the compound is administered at a dose of from about 0.3 mg to about 0.8 mg once daily. In one embodiment, the compound is administered at a dose of from about 0.3 mg to about 1 mg once daily. In one embodiment, the compound is administered at a dose of from about 0.6 mg to about 0.8 mg once daily. In one embodiment, the compound is administered at a dose of from about 0.6 mg to about 1 mg once daily. In one embodiment, the compound is administered at a dose of from about 0.8 mg to about 1 mg once daily.

In one embodiment, a compound described herein, e.g., Compound 1, or pharmaceutically acceptable salt thereof (e.g., a hydrobromide salt of Compound 1), is administered at a dose of about 0.1 mg, about 0.15 mg, about 0.2 mg, about 0.3 mg, about 0.4 mg, about 0.45 mg, about 0.5 mg, about 0.6 mg, about 0.7 mg, about 0.8 mg, about 0.9 mg, about 1 mg, about 1.1 mg, about 1.2 mg, about 1.3 mg, about 1.4 mg, about 1.5 mg, about 1.6 mg, about 1.7 mg, about 1.8 mg, about 1.9 mg, or about 2 mg once daily. In one embodiment, the compound is administered at a dose of about 0.1 mg once daily. In one embodiment, the compound is administered at a dose of about 0.15 mg once daily. In one embodiment, the compound is administered at a dose of about 0.2 mg once daily. In one embodiment, the compound is administered at a dose of about 0.3 mg once daily. In one embodiment, the compound is administered at a dose of about 0.4 mg once daily. In one embodiment, the compound is administered at a dose of about 0.45 mg once daily. In one embodiment, the compound is administered at a dose of about 0.5 mg once daily. In one embodiment, the compound is administered at a dose of about 0.6 mg once daily. In one embodiment, the compound is administered at a dose of about 0.7 mg once daily. In one embodiment, the compound is administered at a dose of about 0.8 mg once daily. In one embodiment, the compound is administered at a dose of about 0.9 mg once daily. In one embodiment, the compound is administered at a dose of about 1 mg once daily. In one embodiment, the compound is administered at a dose of about 1.1 mg once daily. In one embodiment, the compound is administered at a dose of about 1.2 mg once daily. In one embodiment, the compound is administered at a dose of about 1.3 mg once daily. In one embodiment, the compound is administered at a dose of about 1.4 mg once daily. In one embodiment, the compound is administered at a dose of about 1.5 mg once daily. In one embodiment, the compound is administered at a dose of about 1.6 mg once daily. In one embodiment, the compound is administered at a dose of about 1.7 mg once daily. In one embodiment, the compound is administered at a dose of about 1.8 mg once daily. In one embodiment, the compound is administered at a dose of about 1.9 mg once daily. In one embodiment, the compound is administered at a dose of about 2 mg once daily.

In one embodiment, a compound described herein, e.g., Compound 1, or pharmaceutically acceptable salt thereof (e.g., a hydrobromide salt of Compound 1), is administered for 7 days followed by 7 days of rest. In one embodiment, the compound is administered for 14 days followed by 7 days of rest. In one embodiment, the compound is administered for 21 days followed by 7 days of rest.

In one embodiment, a compound described herein, e.g., Compound 1, or pharmaceutically acceptable salt thereof (e.g., a hydrobromide salt of Compound 1), is administered on days 1 to 14 of a 21-day cycle. In one embodiment, the compound is administered on days 1 to 21 of a 28-day cycle. In one embodiment, the compound is administered on days 1 to 7 and days 15 to 21 of a 28-day cycle. The administration period of the compound is followed by rest of the compound on the remaining days of the cycle.

In one embodiment, provided herein is a method for treating relapsed or refractory multiple myeloma, comprising (i) administering elotuzumab on days 1, 8, 15, and 22 of first two 28-day cycles, and on day 1 of subsequent 28-day cycle(s); (ii) administering dexamethasone on days 1, 8, 15, and 22 of each of the 28-day cycles; and (iii) administering the compound on days 1 to 21 of each of the 28-day cycles.

In one embodiment, provided herein is a method for treating relapsed or refractory multiple myeloma, comprising (i) administering isatuximab on days 1, 8, 15, and 22 of a first 28-day cycle, and on days 1 and 15 of subsequent 28-day cycle(s); (ii) administering dexamethasone on days 1, 8, 15, and 22 of each of the 28-day cycles; and (iii) administering the compound on days 1 to 21 of each of the 28-day cycles.

In one embodiment, provided herein is a method for treating relapsed or refractory multiple myeloma, comprising (i) administering isatuximab on days 1, 8, 15, and 22 of a first 28-day cycle, and on days 1 and 15 of subsequent 28-day cycle(s); (ii) administering dexamethasone on days 1, 8, 15, and 22 of each of the 28-day cycles; and (iii) administering the compound on days 1 to 7 and days 15 to 21 of each of the 28-day cycles.

In one embodiment, provided herein is a method for treating relapsed or refractory multiple myeloma, comprising (i) administering bortezomib on days 1, 4, 8 and 11 of first eight 21-day cycles, and on days 1 and 8 of subsequent 21-day cycle(s); (ii) administering dexamethasone on days 1, 2, 4, 5, 8, 9, 11 and 12 of first eight 21-day cycles, and on days 1, 2, 8 and 9 of subsequent 21-day cycle(s); and (iii) administering the compound on days 1 to 14 of each of the 21-day cycles.

In one embodiment, provided herein is a method for treating relapsed or refractory multiple myeloma, comprising (i) administering daratumumab on days 1, 8, 15, and 22 of first two 28-day cycles, on days 1 and 15 of the third to sixth 28-day cycles, and on day 1 of subsequent 28-day cycle(s); (ii) administering dexamethasone on days 1, 8, 15, and 22 of each of the 28-day cycles; and (iii) administering the compound on days 1 to 21 of each of the 28-day cycles.

In one embodiment, provided herein is a method for treating relapsed or refractory multiple myeloma, comprising (i) administering daratumumab on days 1, 8, and 15 of first three 21-day cycles, on day 1 of the fourth to eighth 21-day cycles, and on day 1 of subsequent 28-day cycle(s); (ii) administering dexamethasone on days 1, 8, and 15 of first eight 21-day cycles, and on days 1, 8, 15, and 22 of subsequent 28-day cycle(s); and (iii) administering the compound on days 1 to 14 of first eight 21-day cycles, and on days 1 to 21 of subsequent 28-day cycle(s).

In one embodiment, provided herein is a method for treating relapsed or refractory multiple myeloma, comprising (i) administering daratumumab on days 1, 8, 15, and 22 of first two 28-day cycles, on days 1 and 15 of the third to sixth 28-day cycles, and on day 1 of subsequent 28-day cycle(s); (ii) administering dexamethasone on days 1, 8, 15, and 22 of each of the 28-day cycles; and (iii) administering the compound on days 1 to 7 and days 15 to 21 of first six 28-day cycles, and on days 1 to 21 on subsequent 28-day cycle(s).

In one embodiment, provided herein is a method for treating relapsed or refractory multiple myeloma, comprising (i) administering carfilzomib on days 1, 8, and 15 of first twelve 28-day cycles, and on days 1 and 15 of subsequent 28-day cycle(s); (ii) administering dexamethasone on days 1, 8, 15, and 22 of each of the 28-day cycles; and (iii) administering the compound on days 1 to 21 of each of the 28-day cycles.

In one embodiment, provided herein is a method for treating newly diagnosed multiple myeloma, comprising (i) administering bortezomib on days 1, 4, 8 and 11 of (up to) six 21-day cycles; (ii) administering dexamethasone on days 1, 2, 4, 5, 8, 9, 11 and 12 of each of the 21-day cycles; and (iii) administering the compound on days 1 to 14 of each of the 21-day cycles.

### Pharmaceutical Compositions

The pharmaceutical compositions provided herein contain therapeutically effective amounts of one or more of compounds provided herein and/or a second active agent provided herein, and optionally a pharmaceutically acceptable carrier, diluent or excipient.

The compounds can be formulated into suitable pharmaceutical preparations such as solutions, suspensions, tablets, dispersible tablets, pills, capsules, powders, sustained release formulations or elixirs, for oral administration or in sterile solutions or suspensions for ophthalmic or parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Seventh Edition 1999).

In the compositions, effective concentrations of one or more compounds or pharmaceutically acceptable salts is (are) mixed with a suitable pharmaceutical carrier or vehicle. In certain embodiments, the concentrations of the compounds in the compositions are effective for delivery of an amount, upon administration, that treats, prevents, or ameliorates one or more of the symptoms and/or progression of multiple myeloma.

Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, tissue distribution, inactivation, metabolism and excretion rates of the active compound, the physicochemical characteristics of the compound, the dosage schedule, and amount administered as well as other factors known to those of skill in the art. For example, the amount that is delivered is sufficient to ameliorate one or more of the symptoms of cancer, including solid tumors and blood borne tumors.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, pens, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil water emulsions containing suitable quantities of the compounds or pharmaceutically acceptable salts thereof. The pharmaceutically therapeutically active compounds and salts thereof are formulated and administered in unit dosage forms or multiple dosage forms. Unit dose forms as used herein refer to physically discrete units suitable for human and animal subjects and packaged individually as is known in the art. Each unit dose contains a predetermined quantity of the therapeutically active compound sufficient to produce the desired therapeutic effect, in association with the required pharmaceutical carrier, vehicle or diluent. Examples of unit dose forms include ampules and syringes and individually packaged tablets or capsules. Unit dose forms may be administered in fractions or multiples thereof. A multiple dose form is a plurality of identical unit dosage forms packaged in a single container to be administered in segregated unit dose form. Examples of multiple dose forms include vials, bottles of tablets or capsules or bottles of pints or gallons. Hence, multiple dose form is a multiple of unit doses which are not segregated in packaging.

Dosage forms or compositions containing active ingredient in the range of 0.005% to 100% with the balance made up from non toxic carrier may be prepared. For oral administration, a pharmaceutically acceptable non toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art.

The active compounds or pharmaceutically acceptable salts may be prepared with carriers that protect the compound against rapid elimination from the body, such as time release formulations or coatings.

The compositions may include other active compounds to obtain desired combinations of properties. The compounds provided herein, or pharmaceutically acceptable salts thereof as described herein, may also be advantageously administered for therapeutic or prophylactic purposes together with another pharmacological agent known in the general art to be of value in treating one or more of the diseases or medical conditions referred to hereinabove, such as diseases related to oxidative stress. It is to be understood that such combination therapy constitutes a further aspect of the compositions and methods of treatment provided herein.

Certain pharmaceutical compositions and formulations of Compound 1 are described in U.S. Patent Application Publication No. 2020-0215061 and U.S. Application No. 63/048,998, the entirety of which is incorporated herein by reference.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### EXAMPLES

Certain embodiments of the invention are illustrated by the following non-limiting examples.

### Example 1: Phase 1/2 Clinical Study

A Phase 1/2 multicenter, open-label, study is conducted to determine the recommended dose and regimen, and evaluate the safety and preliminary efficacy of Compound 1 in combination with standard treatments in subjects with relapsed or refractory multiple myeloma (RRMM) and newly diagnosed multiple myeloma (NDMM).

### Indication

Relapsed or refractory multiple myeloma (RRMM) and newly diagnosed multiple myeloma (NDMM).

### Objectives

Primary objective: To determine the recommended dose and regimen, and evaluate the safety and preliminary efficacy of Compound 1 in combination with standard treatments in subjects with RRMM and NDMM.

Secondary objective: To evaluate additional measures of efficacy (time-to-response, duration of response, very good partial response [VGPR] or better and complete response rates) of Compound 1 in combination with standard treatments in subjects with RRMM and NDMM.

### Exploratory Objectives:

Assess the PK of Compound 1 when given in combination with standard treatments in subjects with RRMM and NDMM.

Assess the relationship between pharmacokinetics (PK)/pharmacodynamic (PD) biomarkers and clinical outcomes of Compound 1 when given in combination with standard treatments in subjects with RRMM and NDMM.

Evaluate the percentage of subjects who attain minimal residual disease (MRD) negative status by flow cytometry (EuroFlow™).

Evaluate additional measures of efficacy (progression-free survival) of Compound 1 in combination with standard treatments in subjects with RRMM and NDMM.

### Study Design

This is an open-label, multicenter, Phase 1/2 study to determine the maximum tolerated dose (MTD)/recommended phase 2 dose (RP2D) and to evaluate the safety and preliminary efficacy of Compound 1 in combination with standard treatments.

### Phase 1 (˜108 Subjects)

A Modified Toxicity Probability Interval-2 (mTPI-2) design (Ji et al., Clin. Trials. 2010, 7(6):653-63; Ji et al., J. Clin. Oncol. 2013, 31(14): 1785-91; Guo et al., Contemp. Clin. Trials. 2017, 58:23-33) is used to determine the RP2D for Compound 1 in combination with standard treatments in subjects with RRMM who have received 2 to 4 prior regimens. Each cohort acts independently.
Cohort A: Compound 1 in combination with bortezomib (BTZ) and dexamethasone (dex)
Cohort B: comprised of 3 Subcohorts (B1, B2, and B3) of Compound 1 in combination with daratumumab (DARA) and dex; the term Cohort B refers to all Subcohorts B1, B2, and B3.
Cohort C: Compound 1 in combination with carfilzomib (CFZ) and dex
Cohort H: Compound 1 in combination with elotuzumab (ELO) and dex
Cohort I: Compound 1 in combination with isatuximab (ISA) and dex

All subjects within Cohort A and Subcohort B2 are observed for 21 days (Cycle 1) after the first dose of Compound 1, while subjects in Subcohorts B1, B3, and Cohorts C, H, and I are observed for 28 days (Cycle 1) after the first dose of Compound 1 before initiation of the next dose level. All subjects on these Phase 1 treatment cohorts continue study treatment until progressive disease (PD), death, unacceptable toxicity or withdrawal of consent.

The target toxicity rate of dose-limiting toxicity (DLT) for the combination of Compound 1 plus standard treatments is 25% for all schedules (i.e., target toxicity level [TTL] is 0.25). Subjects are enrolled in cohorts of size ≥3 with maximum sample size of 9 for each dose level. The dose levels of Compound 1 for the Phase 1 cohorts are 0.3 mg, 0.6 mg, and 1.0 mg. Based on the data from Subcohort B1, the initial dose level of Compound 1 (0.3 mg or 0.6 mg) is determined for the Phase 1 Subcohorts B2 and B3.

If 1.0 mg Compound 1 is not tolerated, 0.8 mg is tested. For Cohort I, the initial dose level is the RP2D-1 level determined in Subcohorts B1, B2, or B3. When escalating the Compound 1 dose, the maximum dose increment between 2 dose levels is 100% and the maximum planned dose is 1.0 mg. Dose escalation/de-escalation is according to the mTPI-2 algorithm (Ji, 2010; Ji, 2013; Guo, 2017).

The MTD may be the RP2D, however a RP2D below the MTD may also be determined by PK, PD data as well as the safety and preliminary efficacy data, as applicable.

### Phase 2

The safety and preliminary efficacy of Compound 1 in combination with standard treatments in subjects with RRMM and NDMM are further evaluated in the Phase 2 part.

### Part 1 (˜231 Subjects)

Once the RP2D is confirmed for the respective Compound 1 triplet regimen the following cohorts are enrolled at the RP2D (each cohort acting independently):
Cohort D: Compound 1 in combination with BTZ and dex in subjects with RRMM that have received 1 to 3 prior regimens. This cohort enrolls approximately 47 subjects.
Cohort E: Cohort E contains up to 3 Subcohorts (E1, E2, and E3) of Compound 1 in combination with DARA and dex in subjects with RRMM who have received 1 to 3 prior regimens. Each Subcohort starts once the MTD/RP2D in the corresponding Subcohort B is declared and it is deemed that further investigation of that Subcohort is needed. Cohort E enrolls approximately 49 subjects in total with the number of subjects enrolled in each Subcohort determined by the review of available safety data, efficacy data, and as applicable, PK and PD data. The term Cohort E refers to all Subcohorts E1, E2 and E3.
Cohort F: Compound 1 in combination with CFZ and dex in subjects with RRMM that have received 1 to 3 prior regimens. This cohort enrolls approximately 37 subjects.
Cohort J: Compound 1 in combination with ELO and dex in subjects with RRMM that have received ≥2 prior regimens. This cohort enrolls approximately 50 subjects.
Cohort K: Compound 1 in combination with ISA and dex in subjects with RRMM that have received ≥2 prior regimens. This cohort enrolls approximately 48 subjects.

### Part 2 (˜69 Subjects)

If the threshold for minimum ≥VGPR rate for Cohort D is met, an additional cohort is opened for enrollment:
Cohort G: Compound 1 in combination with BTZ and dex for 4 to 6 cycles as induction followed by an autologous stem cell transplant (ASCT) with or without maintenance as part of standard of care (SOC). This cohort enrolls up to 69 transplant-eligible (TE) NDMM subjects.

The study is conducted in compliance with International Conference on Harmonisation (ICH) Good Clinical Practices (GCPs).

### Study Population

The study population consists of subjects with RRMM (Cohorts A, B, C, D, E, F, H, I, J, and K). Cohort G subjects include subjects with NDMM who are eligible for an ASCT.

### Length of Study

The study consists of the following consecutive periods: Screening, Treatment and Follow-up. The Screening period may not exceed a 28-day window prior to start of study treatment (Cycle 1 Day 1). The Treatment period consists of 21-day cycles for Cohorts A, Subcohorts B2, E2 (from Cycles 1 to 8), Cohorts D and G and of 28-day cycles for Subcohorts B1, E1, B2 (from Cycle 9 onwards), B3, E3 and Cohorts C, F, H, I, J, and K. Treatment continues until progressive disease (PD), death, unacceptable toxicity, or withdrawal of consent for all cohorts except Cohort G where treatment continues for up to 6 cycles or until PD, death, unacceptable toxicity or withdrawal of consent before 6 cycles. All subjects have an End of Treatment (EOT) Visit to collect safety and efficacy assessments. For subjects in Cohort G, the EOT Visit is considered either 3 months (±7 days) post ASCT (prior to any maintenance therapy, if applicable) or at any other moment for treatment discontinuation. Another visit is conducted 28 (±3) days after the EOT visit to collect safety assessments.

Subjects who discontinue study treatment for any reason, other than PD or withdrawal of consent, are followed for response assessment every 21 days (for Cohorts A and D) or every 28 days (for Cohorts B, C, E, F, H, I, J, and K) until PD or until a subsequent anti-myeloma regimen has been started whereby a progression-free survival (PFS) Discontinuation Visit is performed. Additionally, subjects in Cohort G, following induction, ASCT with or without maintenance, are followed for response assessment during the PFS follow-up every 3 months until PD or until a subsequent anti-myeloma regimen has been started whereby a PFS Discontinuation Visit is performed.

The End of Trial is defined as either the date of the last visit of the last subject to complete the post-treatment follow-up, or the date of receipt of the last data point from the last subject that is required for primary, secondary and/or exploratory analysis, as prespecified in the protocol, whichever is the later date.

### Study Treatments

For subjects enrolled to Cohorts A, D and G (Compound 1+BTZ+dex):
Oral Compound 1 at specified cohort dose (for Cohort A) or at RP2D (for Cohorts D and G) from Days 1 to 14 of a 21-day cycle
BTZ administered subcutaneously (SC) at a starting dose of 1.3 mg/m² for:
   Cycles 1 to 8 on Days 1, 4, 8 and 11 of a 21-day cycle (up to Cycle 6 for Cohort G)
   Cycles ≥9 on Days 1 and 8 of a 21-day cycle (excluding Cohort G)
Oral dex dosed at 20 mg/day (≤75 years old) or 10 mg/day (>75 years old) for:
   1 to 8 on Days 1, 2, 4, 5, 8, 9, 11 and 12 of a 21-day cycle (up to Cycle 6 for Cohort G)
   Cycles ≥9 on Days 1, 2, 8 and 9 of a 21-day cycle (excluding Cohort G)

For subjects enrolled to Subcohort B1 and Subcohort E1 (Compound 1+DARA+dex):
Oral Compound 1 at specified cohort dose (for Subcohort B1) or at RP2D (for Subcohort E1) from Days 1 to 21 of a 28-day cycle
Either intravenous (IV) DARA administered at a dose of 16 mg/kg or subcutaneous (SC) DARA administered at a dose of 1800 mg over 3 to 5 minutes.
   Cycles 1 and 2 on Days 1, 8, 15, and 22 of a 28-day cycle
   Cycles 3 to 6 on Days 1 and 15 of a 28-day cycle
   Cycles ≥7 on Day 1 of a 28-day cycle
Oral/IV dex administered at a total dose of 40 mg weekly-Days 1, 8, 15 and 22. For subjects older than 75 years or underweight (body mass index [BMI]<18.5), the dex dose may be administered at a dose of 20 mg weekly.
   On days when subjects receive an infusion of DARA, dex is not self-administered but instead is administered at the site. In this setting, dex is utilized as the treatment dose of steroid for that particular day, as well as the required premedication prior to DARA infusion.
Each subject's dose is calculated based on the subject's weight rounded to the nearest kilogram. There is no cap on the absolute dose allowed, as long as the dose does not exceed 16 mg/kg. If a subject's weight changes by more than 10% from baseline, the dose of DARA is re-calculated.

For subjects enrolled to Subcohort B2 and Subcohort E2:
Oral Compound 1 at specified cohort dose (for Subcohort B2) or at RP2D (for Subcohort E2) from Days 1 to 14 of a 21-day cycle from Cycle 1 to Cycle 8 and from Days 1 to 21 of a 28-day cycle from Cycle 9 onwards
Either IV DARA administered at a dose of 16 mg/kg or SC DARA administered at a dose of 1800 mg over 3 to 5 minutes:
   Cycles 1 to 3 on Days 1, 8, 15 of a 21-day cycle
   Cycles 4 to 8 on Day 1 of a 21-day cycle
   Cycles ≥9 on Day 1 of a 28-day cycle
Oral/IV dex administered at a total dose of 40 mg weekly
   Cycles 1 to 8 on Days 1, 8, and 15 of a 21-day cycle
   Cycles ≥9 on Days 1, 8, 15, and 22 of a 28-day cycle
   For subjects older than 75 years or underweight (BMI<18.5), the dex dose may be administered at a dose of 20 mg weekly.
   On days when subjects receive an infusion of DARA, dex is not self-administered but instead is administered at the site. In this setting, dex is utilized as the treatment dose of steroid for that particular day, as well as the required premedication prior to DARA infusion.
Each subject's dose is calculated based on the subject's weight rounded to the nearest kilogram. There is no cap on the absolute dose allowed, as long as the dose does not exceed 16 mg/kg. If a subject's weight changes by more than 10% from baseline, the dose of DARA is re-calculated.

For subjects enrolled to Subcohort B3 and Subcohort E3:
Oral Compound 1 at specified cohort dose (for Subcohort B3) or at RP2D (for Subcohort E3) from Days 1 to 7 and Days 15 to 21 of a 28-day cycle from Cycle 1 to Cycle 6 and from Days 1 to 21 of a 28-day cycle from Cycle 7 onwards.
Either IV DARA administered at a dose of 16 mg/kg or SC DARA administered at a dose of 1800 mg over 3 to 5 minutes.
   Cycles 1 and 2 on Days 1, 8, 15, and 22 of a 28-day cycle
   Cycles 3 to 6 on Days 1 and 15 of a 28-day cycle
   Cycles≥7 on Day 1 of a 28-day cycle
Oral/IV dex is administered at a total dose of 40 mg weekly on Days 1, 8, 15, and 22. For subjects older than 75 years or underweight (BMI<18.5), the dex dose may be administered at a dose of 20 mg weekly.
   On days when subjects receive an infusion of DARA, dex is not self-administered but instead is administered at the site. In this setting, dex is utilized as the treatment dose of steroid for that particular day, as well as the required premedication prior to DARA infusion.
Each subject's dose is calculated based on the subject's weight rounded to the nearest kilogram. There is no cap on the absolute dose allowed, as long as the dose does not exceed 16 mg/kg. If a subject's weight changes by more than 10% from baseline, the dose of DARA is re-calculated.

For subjects enrolled to Cohorts C and F (Compound 1+CFZ+dex):
Oral Compound 1 at specified cohort dose (for Cohort C) or at RP2D (for Cohort F) from Days 1-21 of a 28-day cycle
Intravenous CFZ administered over 30 minutes:
   Cycle 1 (20 mg/m²) on Day 1
   Cycle 1 (56 mg/m²) on Days 8 and 15 (Cycle 1) of a 28-day cycle
   Cycles 2 to 12 (56 mg/m²) on Days 1, 8, and 15 of a 28-day cycle
   Cycles≥13 (56 mg/m²) on Days 1 and 15 of a 28-day cycle
Oral/IV dex dosed at 40 mg/day (20 mg/day for subjects >75 years old) on Days 1, 8, 15, and 22 of a 28-day cycle

For subjects enrolled to Cohorts H and J (Compound 1+ELO+dex):
Oral Compound 1 at specified cohort dose (for Cohort H) or at RP2D (for Cohort J) from Days 1-21 of a 28-day cycle
Intravenous ELO
   Cycles 1 to 2 (10 mg/kg) on Days 1, 8, 15, and 22 of a 28-day cycle
   Cycles ≥3 (20 mg/kg) on Day 1 of a 28-day cycle
Oral/IV dexamethasone
   On ELO dosing days: Oral (28 mg)/IV (8 mg) dex dosed for a total of 36 mg/day (For subjects >75 years old, oral [8 mg]/IV [8 mg]) dex dosed for a total of 16 mg/day)
      Cycles 1 to 2 on Days 1, 8, 15, and 22 of a 28-day cycle
      Cycles ≥3 on Day 1 of a 28-day cycle
   On non-ELO dosing days: Oral dex dosed at 40 mg/day (20 mg/day for subjects >75 years old)
      Cycles ≥3 on Days 8, 15, and 22 of a 28-day cycle

For subjects enrolled to Cohorts I and K (Compound 1+ISA+dex):
Compound 1 dose and schedule in Cohorts I and K is determined based on the data from Subcohorts B1, B2, and B3, given that both DARA and ISA are CD38-directed cytolytic antibodies.
Oral Compound 1 at specified cohort dose (for Cohort I) or at RP2D (for Cohort K) from Days 1 to 21 of a 28-day cycle (21/28 dosing schedule), or from Days 1 to 7 and Days 15 to 21 of a 28-day cycle (14/28 dosing schedule).
IV ISA
   Cycle 1 (10 mg/kg) on Days 1, 8, 15, and 22 of a 28-day cycle
   Cycles ≥2 (10 mg/kg) on Days 1 and 15 of a 28-day cycle
Oral/IV dex administered at a total dose of 40 mg weekly-Days 1, 8, 15 and 22. For subjects older than 75 years the dex dose may be administered at a dose of 20 mg weekly.
   On days when subjects receive an infusion of ISA, dex is not self-administered but instead is administered at the site. In this setting, dex is utilized as the treatment dose of steroid for that particular day, as well as the required premedication prior to ISA infusion.

### Inclusion Criteria

Subjects must satisfy the following criteria to be enrolled in the study:
1. Subject is ≥18 years of age at the time of signing the informed consent form (ICF).
2. Subject must understand and voluntarily sign an ICF prior to any study-related assessments/procedures being conducted.
3. Subject is willing and able to adhere to the study visit schedule and other protocol requirements.
4. Subject has an Eastern Cooperative Oncology Group (ECOG) performance status score of 0, 1 or 2.
5. Females of childbearing potential (FCBP) must:
   a. Have 2 negative pregnancy tests as verified by the Investigator prior to starting study therapy. She must agree to ongoing pregnancy testing during the course of the study, and after end of study treatment. This applies even if the subject practices true abstinence* from heterosexual contact.
   b. Either commit to true abstinence* from heterosexual contact (which must be reviewed on a monthly basis and source documented) or agree to use, and be able to comply with two reliable forms of contraception as defined in the Pregnancy Prevention Plan (PPP) without interruption, 28 days prior to starting Compound 1, during the study treatment (including during dose interruptions), and for 28 days after the last dose of Compound 1, 7 months after the last dose of BTZ (for Cohorts A, D and G), 90 days after the last dose of DARA (for Cohorts B and E), 6 months after the last dose of CFZ or ELO (for Cohorts C and F and Cohorts H and J), or 5 months after the last dose of ISA (for Cohorts I and K), whichever is later.
Note: A female of childbearing potential (FCBP) is a female who: 1) has achieved menarche at some point and, 2) has not undergone a hysterectomy or bilateral oophorectomy, or 3) has not been naturally postmenopausal (amenorrhea following cancer therapy does not rule out childbearing potential) for at least 24 consecutive months (ie, has had menses at any time in the preceding 24 consecutive months).
6. Male subjects must:
   a. Practice true abstinence* (which must be reviewed on a monthly basis) or agree to use of a condom during sexual contact with a pregnant female or a female of childbearing potential while participating in the study (even during dose interruptions) and for at least 3 months after the last dose of Compound 1, DARA (for Cohorts B and E), CFZ (for Cohorts C and F) and ISA (for Cohorts I and K), 4 months after the last dose of BTZ (for Cohorts A, D and G) or 6 months after the last dose of elotuzumab, even if he has undergone a successful vasectomy.
      * True abstinence is acceptable when this is in line with the preferred and usual lifestyle of the subject. Periodic abstinence (e.g., calendar, ovulation, symptothermal, post-ovulation methods) and coitus interruptus (withdrawal) are not acceptable methods of contraception.
7. Males must agree to refrain from donating sperm or semen while on study treatment, and for at least 3 months following last dose of Compound 1, DARA, CFZ and ISA, 4 months after the last dose of BTZ or 6 months after the last dose of elotuzumab. Females must refrain from egg cell (ova) donation while on study treatment, and for 28 days after the last dose of Compound 1.
8. All subjects must agree to refrain from donating blood while on study treatment and for 28 days after the last dose of study treatment.
9. All male and female subjects must follow all requirements defined in the Pregnancy Prevention Plan (PPP).

### For Subjects in Cohorts A, B, C, D, E, F, H, I, J, and K the Following Inclusions Will Also Apply:

10. Subject has documented diagnosis of MM and measurable disease, defined as:
   a. M-protein quantities ≥0.5 g/dL by serum protein electrophoresis (sPEP) or ≥200 mg/24-hour urine collection by urine protein electrophoresis (uPEP) and/or
   b. Serum free light chain (FLC) levels >100 mg/L (10 mg/dL) involved light chain and an abnormal kappa/lambda (K/2) ratio in subjects without measurable disease in the serum or urine
11. Subject has received 2 to 4 (for Cohorts A, B, C, H, and I) or 1 to 3 (Cohorts D, E, and F) or ≥2 (Cohorts J and K) prior anti-myeloma regimens. Note: induction with or without hematopoietic stem cell transplant and with or without maintenance therapy is considered as one regimen.
12. Subject has received prior treatment with a lenalidomide-containing regimen for at least 2 consecutive cycles.
13. Subject achieved a response (minimal response [MR] or better) to at least 1 prior treatment regimen.
14. Subject must have documented disease progression during or after their last antimyeloma regimen.
15. For Cohorts J and K:
   a. Subject has also received prior treatment with a proteasome inhibitor (bortezomib, carfilzomib, or ixazomib) given alone or in combination for at least 2 consecutive cycles AND
   b. Subject has failed therapy with lenalidomide and a proteasome inhibitor, given alone or in combination, defined as progression on or within 60 days of treatment, or disease progression within 6 months after achieving at least a partial response.
   c. Subject is refractory (progressed on or within 60 days of treatment) to their last treatment.
16. Cohort F: Prior therapy with a proteasome inhibitor (PI), excluding carfilzomib, is allowed as long as the subject had at least a PR to prior PI therapy, was not removed from PI therapy due to toxicity, and will have at least a 6-month PI treatment-free interval from last dose received until first study treatment (Subjects may receive maintenance therapy with drugs that are not in PI class during this 6-month treatment free interval).

### For Subjects in Cohort G, the Following Inclusions Will Also Apply:

17. Considered by the investigator to be eligible for high-dose chemotherapy and autologous stem cell transplantation (ASCT) according to the institution's criteria based on age, medical history, cardiac and pulmonary status, overall health and condition, co-morbid condition(s), physical examination, and laboratory.
18. Subject must have documented diagnosis with previously untreated symptomatic MM as defined by the criteria below (Rajkumar et al., Mayo Clinic Proc. 2016, 91(1): 101-19):
   MM diagnostic criteria;
      Clonal bone marrow plasma cells ≥10% or biopsy-proven bony or extramedullary plasmacytoma (Clonality should be established by showing κ/λ-light-chain restriction on flow cytometry, immunohistochemistry, or immunofluorescence. Bone marrow plasma cell percentage should preferably be estimated from a core biopsy specimen; in case of a disparity between the aspirate and core biopsy, the highest value should be used.)
      Any one or more of the following myeloma defining events:
         one or more of the following Myeloma-related organ dysfunction (at least one of the following);
         [C] Calcium elevation (serum calcium >0.25 mmol/L [>1 mg/dL] higher than the upper limit of laboratory normal or >2.75 mmol/L (>11 mg/dL))
         [R] Renal insufficiency (serum creatinine >2 mg/dl) [>177 μmol/L] or creatinine clearance <40 ml/min
         [A] Anemia (hemoglobin <10 g/dl or >2 g/dL below the lower limit of laboratory normal)
         [B] Bone lesions (lytic or osteopenic) one or more bone lesions on skeletal radiography, computed tomography (CT), or positron emission tomography (PET)/CT
         one or more of the following biomarkers of malignancy:
         Clonal bone marrow plasma cell percentage*≥60%
         Abnormal serum free light-chain ratio ≥100 (involved kappa) or <0.01 (involved lambda) and involved FLC level must be ≥100 mg/L
         >1 focal lesion detected by magnetic resonance imaging (MRI) (at least 5 mm in size)
AND have measurable disease, as assessed by central laboratory, defined by any of the following:
   Immunoglobulin (Ig)G myeloma: serum M-protein level ≥1.0 g/dL or urine M-protein level ≥200 mg/24 hours; or
   IgA, IgM, IgD, or IgE multiple myeloma: serum M-protein level >0.5 g/dL or urine M-protein level ≥200 mg/24 hours; or
   Light chain multiple myeloma without measurable disease in serum or urine: serum FLC ≥100 mg/L and abnormal kappa lambda (K/2) ratio

### Exclusion Criteria

The presence of any of the following will exclude a subject from enrollment:
1. Subject has any significant medical condition, laboratory abnormality, or psychiatric illness that would prevent the subject from participating in the study.
2. Subject has any condition including the presence of laboratory abnormalities, which places the subject at unacceptable risk if he/she were to participate in the study.
3. Subject has any condition that confounds the ability to interpret data from the study.
4. Subject has any of the following laboratory abnormalities:
   a. Absolute neutrophil count (ANC)<1,000/μL (for Phase 1 without growth factor support for ≥7 days [≥14 days for pegfilgrastim])
   b. Platelet count: <75,000/μL (it is not permissible to transfuse a subject to reach this level)
   c. Hemoglobin <8 g/dL (<4.9 mmol/L)
   d. Creatinine clearance (CrCl)<45 mL/min (<30 mL/min for Cohort G)
   e. Corrected serum calcium >13.5 mg/dl (>3.4 mmol/L)
   f. Serum aspartate aminotransferase (AST) or alanine aminotransferase (ALT)>2.5×ULN
   g. Serum total bilirubin >1.5×ULN or >3.0 mg/dL for subjects with documented Gilbert's syndrome
   h. Prothrombin time (PT)/international normalized ratio (INR)>1.5×ULN or partial thromboplastin time (PTT)>1.5×ULN, (for subjects not receiving therapeutic anticoagulation).
Note: Subjects receiving therapy for a thromboembolic event that occurred >3 months prior to enrollment are eligible as long as they are on a stable regimen of anticoagulation with warfarin, low-molecular weight heparin or other approved therapeutic anticoagulation regimen.
5. Subject has peripheral neuropathy ≥Grade 2
6. Subject with gastrointestinal disease that may significantly alter the absorption of Compound 1.
7. Subject has prior history of malignancies, other than MM, unless the subject has been free of the disease for ≥5 years with the exception of the following non-invasive malignancies:
   Basal cell carcinoma of the skin
   Squamous cell carcinoma of the skin
   Carcinoma in situ of the cervix
   Carcinoma in situ of the breast
   Incidental histologic finding of prostate cancer (T1a or T1b using the TNM [tumor, nodes, metastasis] clinical staging system) or prostate cancer that is curative
8. Subject has plasma cell leukemia, Waldenstrom's macroglobulinemia, POEMS syndrome (polyneuropathy, organomegaly, endocrinopathy, monoclonal protein, and skin changes) or clinically significant amyloidosis.
9. Subject with known central nervous system (CNS) involvement with myeloma.
10. Subject has received immunosuppressive medication within the last 14 days of initiating study treatment. The following are exceptions to this criterion:
   Intranasal, inhaled, topical or local corticosteroid injections (e.g., intra-articular injection).
   Systemic corticosteroids at doses that do not exceed 10 mg/day of prednisone or the equivalent.
   Steroids as premedication for hypersensitivity reactions (e.g., computed tomography [CT] scan premedication).
11. Subject has impaired cardiac function or clinically significant cardiac disease, including any of the following:
   Left ventricular ejection fraction (LVEF)<45% as determined by echocardiogram (ECHO) or multigated acquisition (MUGA) scan at Screening.
   Complete left bundle branch, bifascicular block or other clinically significant abnormal electrocardiogram (ECG) finding at Screening
   A prolongation of QT interval on Screening ECG as defined by repeated demonstration of a QTc interval >470 milliseconds (msec) using Fridericia's QT correction formula; a history of or current risk factors for torsades de pointes (e.g., heart failure, hypokalemia, or a family history of Long QT Syndrome); and concurrent administration of medications that prolong the QT/QTc interval
   Congestive heart failure (New York Heart Association Class III or IV).
   Myocardial infarction within 12 months prior to starting study treatment.
   Unstable or poorly controlled angina pectoris, including the Prinzmetal variant of angina pectoris
   History of severe coronary artery disease, severe uncontrolled ventricular arrhythmias, sick sinus syndrome, pericardial disease or electrocardiogramavidence of acute ischemia or Grade 3 conduction system abnormalities unless subject has a pacemaker
12. Uncontrolled hypertension or uncontrolled diabetes within 14 days prior to enrollment.
13. Concurrent administration of strong CYP3A modulators; concurrent administration of proton-pump inhibitors (e.g., omeprazole, esomeprazole, lansoprazole, pantoprazole)≤2 weeks prior to starting Compound 1.
14. Subject is a female who is pregnant, nursing or breastfeeding, or who intends to become pregnant during the participation in the study.
15. Subject is positive for human immunodeficiency virus (HIV), chronic or active hepatitis B, or active hepatitis A or C.
16. Subject has a history of anaphylaxis or hypersensitivity to thalidomide, lenalidomide, pomalidomide, BTZ (for Cohorts A, D and G), DARA (for Cohorts B and E), CFZ (for Cohorts C and F), ELO (for Cohorts H and J), ISA (for Cohorts I and K), or dexamethasone.
17. Subject has known or suspected hypersensitivity to the excipients contained in the formulation of Compound 1, BTZ (for Cohorts A, D and G), DARA (for Cohorts B and E), CFZ (for Cohorts C and F), ELO (for Cohorts H and J), ISA (for Cohorts I and K), or dexamethasone.
18. Contraindications to the standard treatment regimens, per local prescribing information.
19. Subject is unable or unwilling to undergo protocol required thromboembolism prophylaxis.

### For Subjects in Cohorts A, B, C, D, E, F, H, I, J, and K, the Following Exclusions Will Also Apply:

20. Subject received any of the following within the last 14 days of initiating study treatment:
   a. Plasmapheresis
   b. Major surgery (as defined by the Investigator)
   c. c. Radiation therapy other than local therapy for myeloma associated bone lesions
   d. d. Use of any systemic anti-myeloma drug therapy
21. Cohorts A and D: Subjects who had progression during treatment or within 60 days of the last dose of BTZ or discontinued BTZ due to toxicity.
22. Cohorts B and I: Subjects who had progression during treatment or within 60 days of the last dose of DARA/ISA or discontinued DARA/ISA due to toxicity.
23. Cohort C: Subjects who had progression during treatment or within 60 days of the last dose of CFZ or discontinued CFZ due to toxicity.
24. Cohorts D, E, F, J, and K: Previous treatment with pomalidomide (POM).
25. Cohorts E and K: Previous treatment with DARA or ISA.
26. Cohort F: Previous treatment with CFZ.
27. Subject used any investigational agents within 28 days or 5 half-lives (whichever is longer) of initiating study treatment.
   Study participation for subjects who have received an investigational vaccine (such as an investigational severe acute respiratory syndrome coronavirus 2 [SARS-COV-2] vaccine) will be determined by discussion between the Investigator and Sponsor Medical Monitor.
28. Subject has received previous allogeneic stem cell transplantation or received autologous stem cell transplantation within 12 weeks prior to starting study treatment.
29. Cohorts B, E, I, and K: Subject has known chronic obstructive pulmonary disease (COPD) with a forced expiratory volume in 1 second (FEV1) 50% of predicted normal. Note that forced expiratory testing (FEV1) is required for subjects suspected of having COPD and subjects must be excluded if FEV1 is <50% of predicted normal.
30. Cohorts B, E, I, and K: Subject has known moderate or severe persistent asthma, or currently has uncontrolled asthma of any classification.
31. Cohorts C and F: Subject has mild hepatic impairment defined as elevated bilirubin >1.0 but <1.5×ULN or normal bilirubin with any elevation of AST.
32. Cohort H: Subjects who had progression during treatment or within 60 days of the last dose of ELO or discontinued ELO due to toxicity
33. Cohort J: Previous treatment with ELO

### For Subjects in Cohort G, the Following Exclusion Criteria Will Also Apply

34. Previous treatment with anti-myeloma therapy (does not include radiotherapy, bisphosphonates, or a single short course of steroid [ie, less than or equal to the equivalent of dexamethasone 40 mg/day for 4 days; such a short course of steroid treatment must not have been given within 14 days of initiating study treatment]).

### For Subjects in all Cohorts

35. Severe acute respiratory syndrome coronavirus 2 (SARS-COV-2) infection within 14 days for mild or asymptomatic infections or within 28 days for severe/critical illness prior to enrollment.
   Acute symptoms must have resolved and there must be no sequelae that would place the subject at a higher risk of clinically significant complications from receiving study treatment, based on the Investigator's assessment in consultation with the Sponsor Medical Monitor.

### Overview of Key Efficacy Assessments

Myeloma paraprotein (serum and 24-hour urine)
Serum immunofixation
Serum immunoglobulins
Serum free light chains
Bone marrow aspiration/biopsy
Percent plasma cells in the bone marrow
Radiographic assessments of lytic bone lesions
Extramedullary plasmacytoma (EMP) assessments
Minimal residual disease assessment
Response per International Myeloma Working Group (IMWG) criteria

### Overview of Key Safety Assessments

Adverse events (AEs)
Complete physical examination including vital signs and venous thromboembolism (VTE) monitoring
Clinical laboratory evaluations (hematology, serum chemistry, urinalysis)
Renal function assessments
Electrocardiogram (ECG)
Pregnancy testing/counseling
Concomitant medications and procedures

### Overview of Pharmacokinetic Assessments

PK samples are collected in a sparse sampling scheme for Compound 1 and its R-enantiomer. Exposure-response analyses is conducted, as appropriate, to assist in identification of the Compound 1 RP2D.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A method of treating multiple myeloma, comprising administering to a subject in need thereof a therapeutically effective amount of a compound of Formula (I): 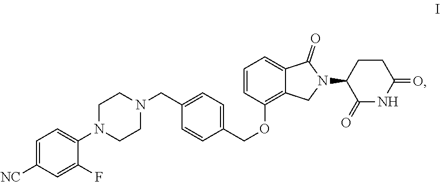 [SMILES: N#Cc1ccc(N2CCN(Cc3ccc(COc4cccc5c4CN([C@H]4CCC(=O)NC4=O)C5=O)cc3)CC2)c(F)c1]
or an enantiomer, mixture of enantiomers, tautomer, isotopolog, or pharmaceutically acceptable salt thereof, in combination with elotuzumab and dexamethasone.

2. The method of claim 1, wherein elotuzumab is administered on days 1, 8, 15, and 22 of first two 28-day cycles, and on day 1 of subsequent 28-day cycle(s); and dexamethasone is administered on days 1, 8, 15, and 22 of each of the 28-day cycles.

3. The method of claim 1 or 2, wherein elotuzumab is administered intravenously, and dexamethasone is administered intravenously or orally.

4. The method of any one of claims 1 to 3, wherein elotuzumab is administered intravenously at a dose of about 10 mg/kg on days 1, 8, 15, and 22 of first two 28-day cycles, and at a dose of about 20 mg/kg on day 1 of subsequent 28-day cycle(s); and dexamethasone is administered both orally at a dose of about 28 mg and intravenously at a dose of about 8 mg on days 1, 8, 15, and 22 of first two 28-day cycles and on day 1 of subsequent 28-day cycle(s) starting on the third 28-day cycle, and is administered orally at a dose of about 40 mg on days 8, 15, and 22 of subsequent 28-day cycle(s) starting on the third 28-day cycle.

5. The method of any one of claims 1 to 3, wherein elotuzumab is administered intravenously at a dose of about 10 mg/kg on days 1, 8, 15, and 22 of first two 28-day cycles, and at a dose of about 20 mg/kg on day 1 of subsequent 28-day cycle(s); and dexamethasone is administered both orally at a dose of about 8 mg and intravenously at a dose of about 8 mg on days 1, 8, 15, and 22 of first two 28-day cycles and on day 1 of subsequent 28-day cycle(s) starting on the third 28-day cycle, and is administered orally at a dose of about 20 mg on days 8, 15, and 22 of subsequent 28-day cycle(s) starting on the third 28-day cycle.

6. A method of treating multiple myeloma, comprising administering to a subject in need thereof a therapeutically effective amount of a compound of Formula (I): 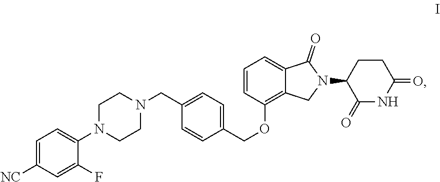 [SMILES: N#Cc1ccc(N2CCN(Cc3ccc(COc4cccc5c4CN([C@H]4CCC(=O)NC4=O)C5=O)cc3)CC2)c(F)c1]
or an enantiomer, mixture of enantiomers, tautomer, isotopolog, or pharmaceutically acceptable salt thereof, in combination with isatuximab and dexamethasone.

7. The method of claim 6, wherein isatuximab is administered on days 1, 8, 15, and 22 of a first 28-day cycle, and on days 1 and 15 of subsequent 28-day cycle(s); and dexamethasone is administered on days 1, 8, 15, and 22 of each of the 28-day cycles.

8. The method of claim 6 or 7, wherein isatuximab is administered intravenously, and dexamethasone is administered intravenously or orally.

9. The method of any one of claims 6 to 8, wherein isatuximab is administered intravenously at a dose of about 10 mg/kg on days 1, 8, 15, and 22 of a first 28-day cycle, and at a dose of about 10 mg/kg on days 1 and 15 of subsequent 28-day cycle(s); and dexamethasone is administered intravenously or orally at a dose of about 40 mg on days 1, 8, 15, and 22 of each of the 28-day cycles.

10. The method of any one of claims 6 to 8, wherein isatuximab is administered intravenously at a dose of about 10 mg/kg on days 1, 8, 15, and 22 of a first 28-day cycle, and at a dose of about 10 mg/kg on days 1 and 15 of subsequent 28-day cycle(s); and dexamethasone is administered intravenously or orally at a dose of about 20 mg on days 1, 8, 15, and 22 of each of the 28-day cycles.

11. The method of any one of claims 1 to 10, wherein the multiple myeloma is relapsed or refractory multiple myeloma (RRMM).

12. The method of claim 11, wherein the subject has received at least two prior lines of therapy.

13. The method of claim 11, wherein the subject has received one to three prior lines of therapy.

14. The method of claim 11, wherein the subject has received two to four prior lines of therapy.

15. The method of any one of claims 12 to 14, wherein the prior lines of therapy include a lenalidomide-containing therapy.

16. The method of any one of claims 12 to 15, wherein the prior lines of therapy include a proteasome inhibitor.

17. The method of claim 16, wherein the proteasome inhibitor is bortezomib, carfilzomib, or ixazomib.

18. The method of any one of claims 1 to 10, wherein the multiple myeloma is newly diagnosed multiple myeloma (NDMM).

19. The method of claim 18, wherein the subject is transplant-eligible.

20. The method of claim 19, wherein the subject is eligible for autologous stem cell transplant (ASCT).

21. The method of any one of claims 1 to 20, wherein a compound of Formula (I), or a pharmaceutically acceptable salt thereof, is administered.

22. The method of claim 21, wherein a compound of Formula (I) is administered.

23. The method of claim 21, wherein a hydrobromide salt of a compound of Formula (I) is administered.

24. The method of any one of claims 1 to 23, wherein the compound is administered orally.

25. The method of any one of claims 1 to 24, wherein the compound is administered at a dose of from about 0.1 mg to about 2 mg once daily.

26. The method of claim 25, wherein the compound is administered at a dose of from about 0.3 mg to about 1 mg once daily.

27. The method of claim 25, wherein the compound is administered at a dose of about 0.3 mg, about 0.6 mg, about 0.8 mg, or about 1 mg once daily.

28. The method of any one of claims 1 to 27, wherein the compound is administered for 7 days followed by 7 days of rest, for 14 days followed by 7 days of rest, or for 21 days followed by 7 days of rest.

29. The method of claim 1, which is for treating relapsed or refractory multiple myeloma, comprising (i) administering elotuzumab on days 1, 8, 15, and 22 of first two 28-day cycles, and on day 1 of subsequent 28-day cycle(s); (ii) administering dexamethasone on days 1, 8, 15, and 22 of each of the 28-day cycles; and (iii) administering the compound on days 1 to 21 of each of the 28-day cycles.

30. The method of claim 6, which is for treating relapsed or refractory multiple myeloma, comprising (i) administering isatuximab on days 1, 8, 15, and 22 of a first 28-day cycle, and on days 1 and 15 of subsequent 28-day cycle(s); (ii) administering dexamethasone on days 1, 8, 15, and 22 of each of the 28-day cycles; and (iii) administering the compound on days 1 to 21 of each of the 28-day cycles.

31. The method of claim 6, which is for treating relapsed or refractory multiple myeloma, comprising (i) administering isatuximab on days 1, 8, 15, and 22 of a first 28-day cycle, and on days 1 and 15 of subsequent 28-day cycle(s); (ii) administering dexamethasone on days 1, 8, 15, and 22 of each of the 28-day cycles; and (iii) administering the compound on days 1 to 7 and days 15 to 21 of each of the 28-day cycles.

32. A compound for use in a method of treating multiple myeloma, wherein the method comprises administering to a subject in need thereof a therapeutically effective amount of the compound characterized by Formula (I): 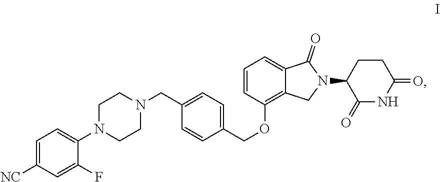 [SMILES: N#Cc1ccc(N2CCN(Cc3ccc(COc4cccc5c4CN([C@H]4CCC(=O)NC4=O)C5=O)cc3)CC2)c(F)c1]
or an enantiomer, mixture of enantiomers, tautomer, isotopolog, or pharmaceutically acceptable salt thereof, in combination with elotuzumab and dexamethasone.

33. The compound for use of claim 32, wherein elotuzumab is administered on days 1, 8, 15, and 22 of first two 28-day cycles, and on day 1 of subsequent 28-day cycle(s); and dexamethasone is administered on days 1, 8, 15, and 22 of each of the 28-day cycles; and/or wherein elotuzumab is administered intravenously, and dexamethasone is administered intravenously or orally.

34. The compound for use of claim 32 or 33, wherein elotuzumab is administered intravenously at a dose of about 10 mg/kg on days 1, 8, 15, and 22 of first two 28-day cycles, and at a dose of about 20 mg/kg on day 1 of subsequent 28-day cycle(s); and dexamethasone is administered both orally at a dose of about 28 mg and intravenously at a dose of about 8 mg on days 1, 8, 15, and 22 of first two 28-day cycles and on day 1 of subsequent 28-day cycle(s) starting on the third 28-day cycle, and is administered orally at a dose of about 40 mg on days 8, 15, and 22 of subsequent 28-day cycle(s) starting on the third 28-day cycle; or
wherein elotuzumab is administered intravenously at a dose of about 10 mg/kg on days 1, 8, 15, and 22 of first two 28-day cycles, and at a dose of about 20 mg/kg on day 1 of subsequent 28-day cycle(s); and dexamethasone is administered both orally at a dose of about 8 mg and intravenously at a dose of about 8 mg on days 1, 8, 15, and 22 of first two 28-day cycles and on day 1 of subsequent 28-day cycle(s) starting on the third 28-day cycle, and is administered orally at a dose of about 20 mg on days 8, 15, and 22 of subsequent 28-day cycle(s) starting on the third 28-day cycle.

35. A compound for use in a method of treating multiple myeloma, wherein the method comprises administering to a subject in need thereof a therapeutically effective amount of the compound characterized by Formula (I): 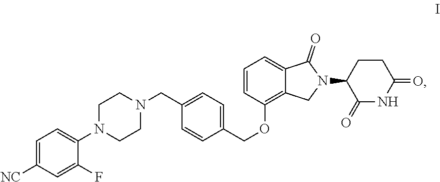 [SMILES: N#Cc1ccc(N2CCN(Cc3ccc(COc4cccc5c4CN([C@H]4CCC(=O)NC4=O)C5=O)cc3)CC2)c(F)c1]
or an enantiomer, mixture of enantiomers, tautomer, isotopolog, or pharmaceutically acceptable salt thereof, in combination with isatuximab and dexamethasone.

36. The compound for use of claim 35, wherein isatuximab is administered on days 1, 8, 15, and 22 of a first 28-day cycle, and on days 1 and 15 of subsequent 28-day cycle(s); and dexamethasone is administered on days 1, 8, 15, and 22 of each of the 28-day cycles; and/or wherein isatuximab is administered intravenously, and dexamethasone is administered intravenously or orally.

37. The compound for use of claim 35 or 36, wherein isatuximab is administered intravenously at a dose of about 10 mg/kg on days 1, 8, 15, and 22 of a first 28-day cycle, and at a dose of about 10 mg/kg on days 1 and 15 of subsequent 28-day cycle(s); and dexamethasone is administered intravenously or orally at a dose of about 40 mg on days 1, 8, 15, and 22 of each of the 28-day cycles; or
wherein isatuximab is administered intravenously at a dose of about 10 mg/kg on days 1, 8, 15, and 22 of a first 28-day cycle, and at a dose of about 10 mg/kg on days 1 and 15 of subsequent 28-day cycle(s); and dexamethasone is administered intravenously or orally at a dose of about 20 mg on days 1, 8, 15, and 22 of each of the 28-day cycles.

38. The compound for use of any one of claims 32 to 37, wherein the multiple myeloma is relapsed or refractory multiple myeloma (RRMM).

39. The compound for use of claim 38, wherein the subject has received at least two prior lines of therapy, wherein the subject has received one to three prior lines of therapy; or wherein the subject has received two to four prior lines of therapy; optionally wherein the prior lines of therapy include a lenalidomide-containing therapy and/or wherein the prior lines of therapy include a proteasome inhibitor.

40. The compound for use of claim 39, wherein the proteasome inhibitor is bortezomib, carfilzomib, or ixazomib.

41. The compound for use of any one of claims 32 to 37, wherein the multiple myeloma is newly diagnosed multiple myeloma (NDMM).

42. The compound for use of claim 41, wherein the subject is transplant-eligible; optionally wherein the subject is eligible for autologous stem cell transplant (ASCT).

43. The compound for use of any one of claims 32 to 43, wherein a compound of Formula (I), or a pharmaceutically acceptable salt thereof, is administered; optionally wherein a compound of Formula (I) is administered.

44. The compound for use of claim 43, wherein a hydrobromide salt of a compound of Formula (I) is administered.

45. The compound for use of any one of claims 32 to 44, wherein the compound is administered orally; and/or wherein the compound is administered at a dose of from about 0.1 mg to about 2 mg once daily; optionally wherein the compound is administered at a dose of from about 0.3 mg to about 1 mg once daily, or wherein the compound is administered at a dose of about 0.3 mg, about 0.6 mg, about 0.8 mg, or about 1 mg once daily.

46. The compound for use of any one of claims 32 to 45, wherein the compound is administered for 7 days followed by 7 days of rest, for 14 days followed by 7 days of rest, or for 21 days followed by 7 days of rest.

47. The compound for use of claim 32, wherein the method is for treating relapsed or refractory multiple myeloma, and the method comprises (i) administering elotuzumab on days 1, 8, 15, and 22 of first two 28-day cycles, and on day 1 of subsequent 28-day cycle(s); (ii) administering dexamethasone on days 1, 8, 15, and 22 of each of the 28-day cycles; and (iii) administering the compound on days 1 to 21 of each of the 28-day cycles.

48. The compound for use of claim 35, wherein the method is for treating relapsed or refractory multiple myeloma, and the method comprises (i) administering isatuximab on days 1, 8, 15, and 22 of a first 28-day cycle, and on days 1 and 15 of subsequent 28-day cycle(s); (ii) administering dexamethasone on days 1, 8, 15, and 22 of each of the 28-day cycles; and (iii) administering the compound on days 1 to 21 of each of the 28-day cycles; or
wherein the method is for treating relapsed or refractory multiple myeloma, and the method comprises (i) administering isatuximab on days 1, 8, 15, and 22 of a first 28-day cycle, and on days 1 and 15 of subsequent 28-day cycle(s); (ii) administering dexamethasone on days 1, 8, 15, and 22 of each of the 28-day cycles; and (iii) administering the compound on days 1 to 7 and days 15 to 21 of each of the 28-day cycles.
